(19) United States
(12) Patent Application Publication
(10) Pub. No.: US 2006/0154261 A1
(43) Pub. Date: Jul. 13, 2006

(54) Method for determining susceptibility of an individual to allergen induced hypersensitivity

(76) Inventors: Andrew Saxon, Santa Monica, CA (US); David-Diaz Sanchez, Canyon City, CA (US); Frank D. Gilliland, Pasadena, CA (US)

Correspondence Address: HELLER EHRMAN LLP, 275 MIDDLEFIELD ROAD, MENLO PARK, CA, 94025-3506, US

(21) Appl. No.: 11/031,822

(22) Filed: Jan. 7, 2005

(51) Int. Cl. C12Q 1/68

(52) U.S. Cl. 435/6

(57) ABSTRACT

The present invention is related generally to a method for screening subjects to determine those subjects more likely to exhibit a hypersensitivity to allergen in the presence of airborne pollutants, such subject having a GSTM1 null genotype and/or a GSTP1 Ile/Ile genotype. This invention also provides novel or improved pharmaceutical compositions and therapeutic strategies for the treatment of immune diseases resulting from inappropriate or unwanted immune response such as allergic reactions.

This invention was made with government support under Grant Nos. AI50495 and ES09581, awarded by the National Institutes of Health. The Government has certain rights in this invention.

### TECHNICAL FIELD

The present invention is related generally to a method for screening subjects to determine those subjects more likely to exhibit an allergic reactivity to allergen in the presence of airborne pollutants. The present invention is also related to the treatment of immune disease, where management of the disease comprises suppressing an inappropriate or unwanted immune response, for example allergic diseases.

### BACKGROUND OF THE INVENTION

Exposure to ambient air pollution is associated with many adverse health effects ranging from increased symptoms of allergic airway disease to increased mortality (Samet et al., Res. Rep Health Eff Inst. 2000, 94:5-79; Pope et al., JAMA 2002 287:1132-41; Dockery et al., N. Engl. J. Med. 1993, 329:1753-59). Research has focused on the effects of ambient particulate pollution and much evidence indicates that particulate pollution is associated with the occurrence of asthma and other airway allergic diseases (McConnell et al., Environ Health Perspect 1999; 107:757-60, Diaz-Sanchez et al., J. Allergy Clin. Immunol. 1999; 104:1183-88, D'Amato, G. Monaldi Arch Chest Dis. 2002; 57:136-40; Pandya et al., Environ. Health Perspect. 2002; 110 (suppl):103-12; Nel et al., Curr Opin Pulm Med 2001; 7:20-26).

Diesel exhaust contains small particles ranging from nanoparticles to coarse particles with mass concentrated in the accumulation mode centered at 0.2 μm in diameter that have high deposition rates in the lung and long residence times in the atmosphere (Barfknecht et al., Dev. Toxicol. Environ Sci 1982; 10:277-94). These primary diesel exhaust particles aggregate into a broad range of sizes and are important contributors to particular matter less than 10 μm in diameter (PM₁₀) and particular matter less than 2.5 μm in diameter (PM_{2.5}). Inhaled diesel exhaust particles can be deposited in the upper and lower respiratory tract and can participate with allergens in starting and exacerbating allergic diseases in the airway (Diaz-Sanchez et al., J. Immunol. 1997; 158:2406-13; Diaz-Sanchez et al., J. Allergy Clin. Immunol 1999; 104:1183-88; Xu GB and Yu, C P, Aerosol Sci Technol 1987 7:117-23; Oppenheim et al., Annu Rev. Immunol 1991; 9:617-48; Diaz-Sanchez et al., J. Allergy Clin Immunol 2001: 106: 1140-46; Delfino R J, Environ Health Perspect 2002 110(suppl):573-89; Kramer et al., Epidemiology 2000: 11:64-70; Sargai et al., Free Radical Biol. Med 1996, 21:199-209).

Environmental tobacco smoke (ETS) exposure also increases the risk for a wide spectrum of adverse health effects including asthma and other allergic disease occurrences (California Environmental California Environmental Protection Agency. Health Effects of Exposure to Environmental Tobacco Smoke. Sacramento, Calif.: California Environmental Protection Agency; 1997; U.S. Environmental Protection Agency. Respiratory Health Effects of Passive Smoking: Lung Cancer and Other Disorders. Washington, D.C.; 1992. Report No.: EPA/600/6-90/006F; U.S. Department of Health and Human Services. The Health Consequences of Involuntary Smoking. Report of the Surgeon General. Washington, D.C.: Public Health Service; 1986. Report No.: DHHS Publication No (PHS) 87-8398.).

Environmental tobacco smoke has been implicated in various diseases including middle ear effusion, bronchitis and pneumonia in children (Johnson et al., Crit. Rev. Toxicol 1990; 20:369-95). Several epidemiological studies have also found associations between household exposure of ETS and airway disease in children (Lodrup et al., Curr Opin Allergy Clin Immunol 2001; 1:139-43; Gilliland et al., Am J Respir Crit Care Med 2001; 163:429-36; Ehrlich et al., J Asthma 2001; 38:239-51).

Tobacco smoke consists of approximately 6,000 known chemical components. Other particulate pollutants resulting from incomplete combustion of organic materials such as diesel exhaust particles (DEP) contain many of the main constituents of ETS such as polyaromatic hydrocarbons (PAHs) and also activate Phase I and II detoxifying enzymes (Barfknecht et al., Devel Toxicol Environ Sci 1982; 10:277-94; Kumagai et al. Free Radical Biol Med 1997; 22:479-87; Hiura et al.,. J Immunol 1999; 163; Soontjens et al., Atmospheric Environment 1997; 31:219-25). Human and murine in vitro and in vivo studies have demonstrated that diesel exhaust particles (DEP) and polyaromatic hydrocarbons (PAHs) can also induce IgE, increase Th2 cytokine production, select against Th1 cytokines and augment histamine release (Kagawa J. Toxicology 2002; 181:349-53; Diaz-Sanchez D, et al., Cur Allergy Asthma Rep 2003; 3:146-52)

These studies have suggested that emergency room visits, asthma symptom severity, and medication usage are all increased if asthmatic children are exposed to parental smoking. (Martinez et al., Pediatrics 1992; 89:21-6; Butz et al., J Asthma 1992; 28:255-64; Evans et al., Am Rev Resp Dis 1987; 135:567-72; Bener et al., Human Biol 1996; 68:405-14). Indeed, the US EPA in its health review document on passive smoking concluded that environmental tobacco smoke was a major cause of childhood asthma exacerbation, that worsened the condition of between 200,000 and one million asthmatic children in the U.S. (Assessment Office of Health and Environment Respiratory health effects of passive smoking: lung cancer and other disorders. Washington, D.C.: US Environmental Protection Agency. 1992.) Most studies have focused on childhood as this is when asthma often begins, environmental tobacco smoke exposure may be assessed more readily and confounders such as active smoking are few. A few studies that have studied adult populations have seen associations between asthma severity and environmental tobacco smoke exposure. Eisner using data obtained from NHANES III showed that ETS is associated with decreased pulmonary function in adults and that this is especially apparent in asthmatics. (Eisner Md. Environ Health Perspect 2002; 110:765-70)

Antioxidants reduce the pro-allergic inflammatory effects of diesel exhaust particles in vitro and in mice (Nel et al., Curr Opin Pulm Med 2001; 7:20-26, Ng et al., J. Immunol. 1998; 9:42-50; Sagai et al., Involvements of superoxide and nitric oxide on asthma-like features induced by diesel exhaust particles in mice: Basel: S. Karger, 1998; Whitekus et al., J. Immunol. 2002 168:2560-67).

Immediate hypersensitivity or Type I allergy is manifested in a broad array of conditions and associated symptoms, which may be mild, chronic, acute and/or life threatening. These various pathologies include, for example, allergic asthma, allergic rhinitis, atopic dermatitis, severe food allergies, chronic urticaria and angioedema, as well as the serious physiological condition of anaphylactic shock. A wide variety of antigens are known to act as allergens, and exposure to these allergens results in the allergic pathology. Common allergens include, but are not limited to, bee stings, penicillin, various food allergies, pollens, animal detritus (especially house dust mite, cat, dog and cockroach), and fungal allergens. The most severe responses to allergens can result in airway constriction and anaphylactic shock, both of which are potentially fatal conditions. Despite advances in understanding the cellular and molecular mechanisms that control allergic responses and improved therapies, the incidence of allergic diseases, especially allergic asthma, has increased dramatically in recent years in both developed and developing countries (Beasley et al., J. Allergy Clin. Immunol. 105:466-472 (2000); Peat and Li, J. Allergy Clin. Immunol. 103:1-10 (1999)).

One object of this invention is to provide novel oand/or improved methods for identifying individuals or patients who are likely to exhibit an enhanced allergic response to allergen in the presence of airborne pollutants.

Another object of this invention is to provide novel or improved therapeutic strategies for the treatment of immune diseases resulting from inappropriate or unwanted immune response such as allergic reactions. The methods for treating allergic diseases provided by the invention can also be used in conjunction with traditional therapies.

### SUMMARY OF THE INVENTION

In brief the invention relates generally to a method for diagnosing or screening individuals or patients to determine those more likely to exhibit allergic reactivity to allergen(s) in the presence of airborne pollutants. This invention also provides novel or improved pharmaceutical compositions and therapeutic strategies for the treatment of immune diseases resulting from inappropriate or unwanted immune response such as allergic reactions. The methods for treating allergic diseases provided by the invention can also be used in conjunction with traditional therapies.

In one aspect the invention is related to a method of determining whether a subject is susceptible to enhancement of allergen-induced hypersensitivity reaction by airborne pollutants comprising genotyping said subject for genes selected from the group consisting of GSTM1 and GSTP1 genes.

It is contemplated that the reaction to the allergen is a hypersensitivity reaction or type I allergic reaction. It is further contemplated that the reaction is an increase in production of immunoglobulin E (IgE) to allergens.

In one aspect the airborne pollutants are selected from the group consisting of traffic related pollutants or indoor pollutants. In another aspect the pollutants are selected from both particulate and gaseous pollutants.

In one aspect the gene is GSTM1. Where the gene is GSTM1 the polymorphism includes GSTM1 null genotype or the GSTM1 present genotype. The GSTM1 present polymorphism is selected from either the short or longer isoform.

In another aspect the gene is GSTP1. Where the gene is GSTP1 the polymorphism includes the GSTP1 105Ile or the GSTP1 105 Val polymorphism. Accordingly, possible genotypes include the GSTP1 105 Ile/Ile genotype, the GSTP1 105 Ile/Val genotype or the GSTP1 105 Val/Val genotype.

In one aspect the screening method is selected from the group consisting of polymerase chain reaction (PCR) analysis, probe hybridization, restriction fragment length polymorphism (RFLP) analysis, minisequencing, MALS-TOF, SINE, heteroduplex analysis, single strand conformational polymorphism (SSCP), denaturing gradient gel electrophoresis (DGGE) and temperature gradient gel electrophoresis (TGGE).

In another aspect the screening method further comprises the step of amplifying the amount of the GSTP1 or GSTM1 gene or a portion thereof which contains said polymorphism. The amplification includes the steps of: selecting a forward and a reverse sequence primer capable of amplifying a region of the GSTP1 or GSTM1 gene which contains a polymorphic site. In another aspect the primers for the GSTP1 gene are selected from the group comprising the sequences: 5′-CCTGGTGGACATGGTGAATG-3′ (SEQ ID NO:6) and 5′TGCTCACACCATAGTTGGTGTAGATGA-3′ (SEQ ID NO:7). In another aspect the primers for the GSTM1 gene are selected from the group comprising the sequences: 5′-CTTGGAGGAACTCCCTGAAAAG-3′ (SEQ ID NO:1) and 5′-TGGAACCTCCATAACACGTGA-3′ (SEQ ID NO:2). In another aspect the genotype is identified with a probe from the GSTM1 gene or from the GSPT1 gene. In one aspect the probe for the GSTM1 gene is AAGCGGCCATGGTTTGCAGG (SEQ ID NO:3). In another aspect the probe for the GSTP1 gene is (TGCAAATACATCTCCCT (allele 1) (SEQ ID NO:9) for GSTP1 105Ile and CTGCAAATACGTCTCC (allele 2) (SEQ ID NO:10) for GSTP1 105Val.

One aspect of the invention concerns a kit for determining whether a subject is susceptible to enhancement of allergen-induced hypersensitivity reaction by airborne pollutants comprising a set of primers to amplify a gene selected from the group consisting of the GSTM1 gene or the GSTP1 gene, DNA polymerase and a probe to identify the GSTM1 polymorphism or the GSTP1 polymorphism respectively.

One aspect of the invention concerns a pharmaceutical composition comprising a protein or polypeptide selected from the group consisting on GSTM1 protein, GSTP1 105val protein, GSTM1 nucleic acid coding sequence or GSTP1 val 105 nucleic acid coding sequence in admixture with a pharmaceutically acceptable excipient or ingredient. In still a further aspect, the invention concerns an article of manufacture comprising a container, a protein or polypeptide selected from the group consisting on GSTM1 protein or GSTP1 105val protein within the container and a label or package insert on or associated with the container. The label or package insert preferably comprises instructions for the treatment or prevention of an immune disease.

In a further aspect, the present invention concerns methods for the treatment and prevention of enhancement of allergen induced hypersensitivity reaction by airborne pollutants in a subject where the subject is administered a polypeptide or protein selected from the group consisting of GSTM1 protein or GSTP1 105val protein or a nucleic acid sequence selected from the group consisting of GSTM1 coding sequence or GSTP1 105 val coding sequence. In one embodiment, the invention concerns a method of treatment comprising administering at least one, or alternatively multiple times, an effective amount of at least one polypeptide or protein selected from the group consisting of GSTM1 protein or GSTP1 105val protein to a subject diagnosed with or at risk of developing enhancement of allergen induced hypersensitivity reaction by airborne pollutants.

In a further aspect of this method of the invention, the therapy received by the subject is received prior to the subject receiving immunotherapy, co-administered to the subject during immunotherapy or administered to the subject after the subject receives immunotherapy.

In a further aspect the invention is directed to a method for detecting a biological effect in subjects exposed to air pollutants comprising genotyping said subjects for polymorphisms within a group of gene comprising GSTM1 and GSTP1. In a further aspect the invention is directed to a method for detecting a modified biological effect in response to exposure to air pollutants in subjects with a GSTM1 null genotype compared to the biological effect detected in subject with a GSTM1 present genotype.

These and other aspects of the invention will become more evident upon reference to the following detailed description and attached drawings. It is to be understood however that various changes, alterations and substitutions may be made to the specific embodiments disclosed herein without departing from their essential spirit and scope. In addition, it is further understood that the drawings are intended to be illustrative and symbolic representations of an exemplary embodiment of the present invention and that other non-illustrated embodiments are within the scope of the invention.

### BRIEF DESCRIPTION OF THE DRAWINGS

FIG. 1 is a graph of the nasal allergen specific IgE response to allergens plus clean air and allergen plus diesel exhaust particles for GSTM1 absent (upper) and present(lower) genotypes. The Y axis is a log scale of median IgE concentrations.

FIG. 2 is a graph of the nasal allergen-specific IgE response to allergens plus clean air and allergen plus environmental tobacco smoke for GSTM1 absent (upper) and present(lower) genotypes. The Y axis is a log scale of median IgE concentrations.

FIG. 3A is the nucleic acid sequence of the human GSTM1 short isoform (SEQ ID NO:11). FIG. 3B is the amino acid sequence of the human GSTM1 short isoform (SEQ ID NO:12).

FIG. 4A is the nucleic acid sequence of the human GSTM1 long variant form (SEQ ID NO:13). FIG. 3B is the amino acid sequence of the human GSTM1 long variant form(SEQ ID NO:14).

FIG. 5A is the nucleic acid sequence of the human GSTP1 105 isoleucine variant (SEQ ID NO: 15). FIG. 5B is the amino acid sequence of the human GSTP1 105 isoleucine variant (SEQ ID NO:16).

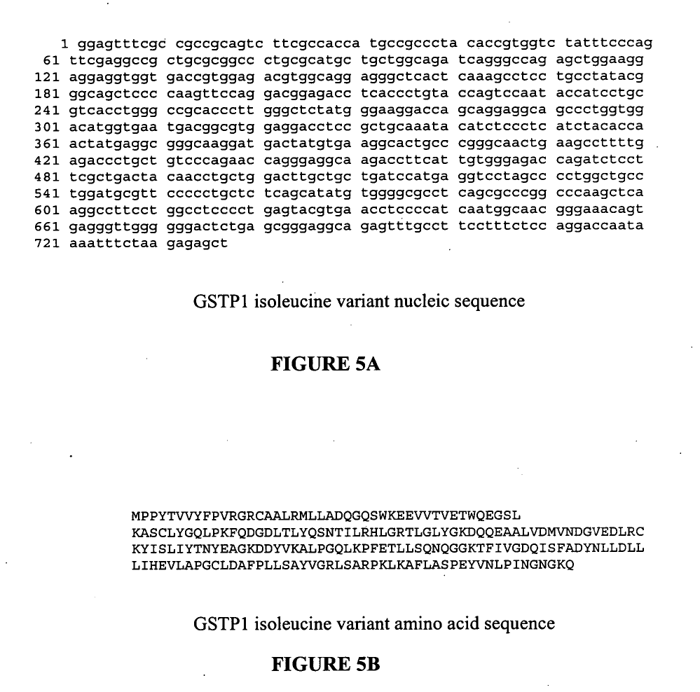

### DETAILED DESCRIPTION OF THE INVENTION

Unless defined otherwise, technical and scientific terms used herein have the same meaning as commonly understood by one of skill in the art to which this invention belongs.

One skilled in the art will recognize many methods and materials similar or equivalent to those described herein, which could be used in the practice of this invention. Indeed the present invention is no way limited to the methods and materials described herein. For purposes of the present invention the following terms are defined.

### I. Definitions

The term “airborne pollutants” means, traffic related pollutants and indoor pollutants. The airborne pollutants can be generated from a number of sources, including but not limited to diesel exhaust and cigarette smoke.

Traffic related pollutants include, but are not limited to, ozone, nitrogen dioxide, diesel exhaust particles and small particulate matter.

“Small particulate matter” means airborne particles ranging in size from nanoparticles to coarse particles that have high deposition rates in the lung and long residence times in the atmosphere. The particles are generally less than 15 μm in diameter, preferably less than 10 μm in diameter, more preferably less than 5 μm and most preferably less than 2.5 μm in diameter. The particulates range in size from 0.1 μm to 15 μm in diameter, preferably from 0.2 μm to 10 μm, more preferably from 0.2 μm to 5 μm, most preferably from 0.2 μm to 2.5 μm.

Indoor pollutants include, but are not limited to, tobacco smoke particles and cooking combustion products.

“Diesel exhaust particles” means respirable airborne pollutants produced during compression ignition of diesel fuel. The particles are composed of elemental and organic carbon compounds as well as trace amount of other elements with toxic properties including transition metals.

“Tobacco smoke particles” means respirable airborne pollutants produced during the smoking of tobacco products including, but not limited to cigarettes and cigars. Such airborne pollutants may be found in the smoke inhaled by the smoker and in environmental tobacco smoke (ETS) or “second hand smoke” (SHS) inhaled by third parties in close proximity to the tobacco smoker.

Glutathione-S-transferases are involved in phase 2 xenobiotic and reactive oxygen species metabolism and have coordinate regulation based on antioxidant response element in their promoter region.

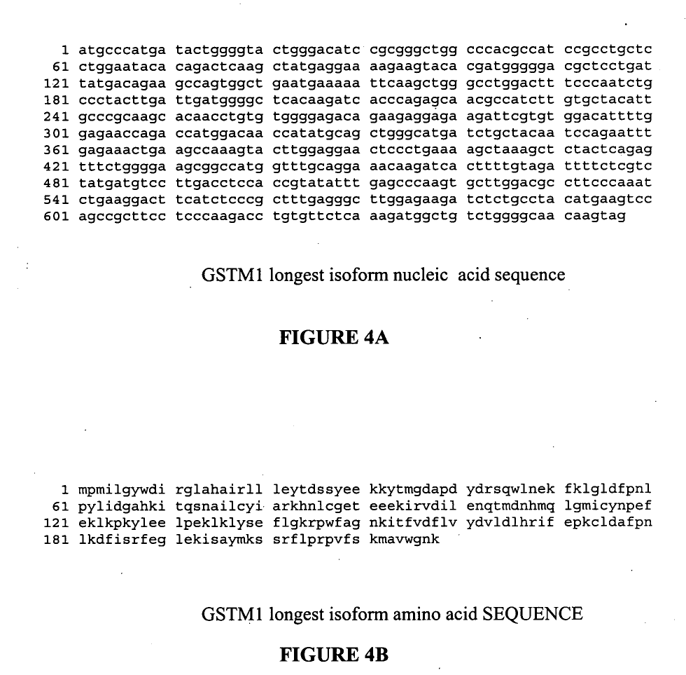

“GSTM1” or “Glutathione-S-transferase M1” is a member of the M family of glutathione-S-transferases. The gene is located at 1p13.3 and has a common allele that results in no protein product (i.e. the “null” allele). (London et al., J. National Cancer Inst. 1995 87:1246-1253; Mattey et al., Ann Rheum Dis 1999 58:164-168) and an allele that provide the functional protein product (the present polymorphism) The GSTM1 null phenotype results from a deletion of the genome so that there is no expression of the GSTM1 protein. Preferably the protein is the human protein. (FIG. 4A, 4B, 5A, 5B)

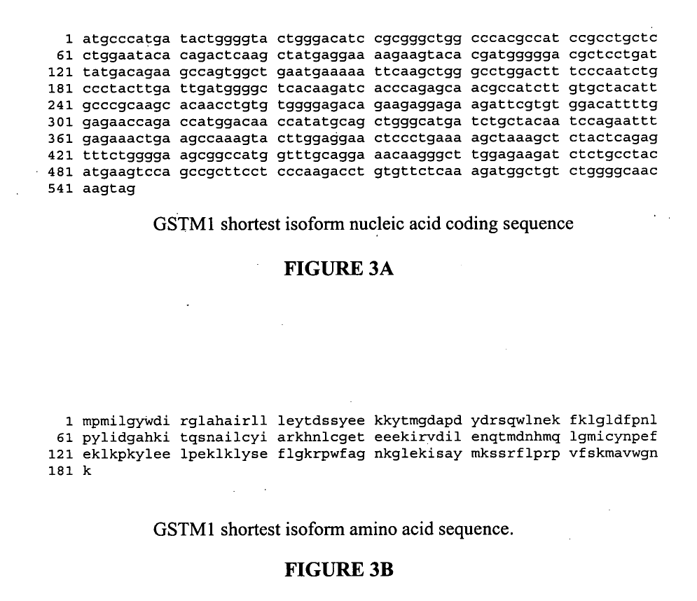

“GSTP1”, “Glutathione-S-transferase P1” is a member of the P family of glutathione-S-transferases. GSTP1 is located at 11q13.3 and has a common single nucleotide polymorphism at codon (A105G) that results in an amino acid change in the protein from isoleucine to valine. This amino acid substitution has pleotropic effects on the enzyme function. (Haries et al., Carcinogenesis 1997 18(4) 641-644; U.S. Pat. No. 5,968,737). Preferably the protein is the human protein. (FIGS. 3A and 3B shows the isoleucine variant of the protein)

“GSTT1” or “Glutathione-S-transferase T1” is a member of the γ family of glutathione-S-transferases. GSTT1 is located 22q11.23 and, like GSTM1 has a common allele that results in no protein product (i.e. the “null” allele).

The term “reactive oxygen species” for biological systems includes, but is not limited to superoxide, hydrogen peroxide, and hydroxyl radical. At low levels, these species may function in cell signaling processes. At higher levels, reactive oxygen species damage cellular macromolecules and participate in apoptotic processes.

The term “mammal” or “mammalian species” refers to any animal classified as a mammal, including humans, domestic and farm animals, and zoo, sports, or pet animals, such as dogs, cats, cattle, horses, sheep, pigs, goats, rabbits, as well as rodents such as mice and rats, etc. Preferably, the mammal is human.

The terms “subject” or “patient,” as used herein, are used interchangeably, and can refer to any to animal, and preferably a mammal, that is the subject of an examination, treatment, analysis, test or diagnosis. In one embodiment, humans are a preferred subject. A subject or patient may or may not have a disease or other pathological condition.

The term “percent (%) nucleic acid sequence identity” with respect to the nucleic acid sequence identified herein is defined as the percentage of nucleotides in a candidate sequence that are identical with the nucleotides in the sequence of interest, after aligning the sequences and introducing gaps, if necessary, to achieve the maximum percent identity. Alignment for the purposes of determining percent nucleic acid sequence identity can be achieved in various ways that are within the skill in the art. Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL® in the PC/Gene program by Intelligenetics, Mountain View, Calif.; GAP®, BESTFIT®, BLAST®, FASTA®, and TFASTA® in the Wisconsin Genetics Software Package, Genetics Computer Group (GCG), 575 Science Dr., Madison, Wis., USA. the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN® for nucleotide query sequences against nucleotide database sequences; BLASTX® for nucleotide query sequences against protein database sequences; BLASTP® for protein query sequences against protein database sequences; TBLASTN® for protein query sequences against nucleotide database sequences; and TBLASTX® for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST® analyses is publicly available, e.g., through the National Center for Biotechnology-Information.

This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

The term “sequence similarity” as used herein, is the measure of nucleic acid sequence identity, as described above, and in addition also incorporates conservative amino acid substitutions.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions see Ausubel et al., Current Protocols in Molecular Biology, Wiley Interscience Publishers 1995).

“Stringent hybridization conditions” or “high stringency conditions” are sequence dependent and will be different with different environmental parameters (e.g., salt concentrations, and presence of organics). Generally, stringent conditions are selected to be about 5° C. to 20° C. lower than the thermal melting point (Tₘ) for the specific nucleic acid sequence at a defined ionic strength and pH. Preferably, stringent conditions are about 5° C. to 10° C. lower than the thermal melting point for a specific nucleic acid bound to a perfectly complementary nucleic acid. The Tₘ is the temperature (under defined ionic strength and pH) at which 50% of a nucleic acid (e.g., tag nucleic acid) hybridizes to a perfectly matched probe.

“Stringent” wash conditions are ordinarily determined empirically for hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal to noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is more important than the measure of any single parameter. See, e.g., Wetmur et al., J. Mol. Biol. 31:349-70 (1966), and Wetmur, Critical Reviews in Biochemistry and Molecular Biology 26(34):227-59 (1991).

In a preferred embodiment, “stringent conditions” or “high stringency conditions,” as defined herein, may be hybridization in 50% formamide, 6×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (100 μg/ml), 0.5% SDS, and 10% dextran sulfate at 42° C., with washes at 42° C. in 2×SSC (sodium chloride/sodium citrate) and 0.1% SDS at 55. ° C., followed by a high-stringency wash consisting of 0.2×SSC containing 0.1% SDS at 42° C.

The terms “complement,” “complementarity” or “complementary,” as used herein, are used to describe single-stranded polynucleotides related by the rules of antiparallel base-pairing. For example, the sequence 5′-CTAGT-3′ is completely complementary to the sequence 5′-ACTAG-3′. Complementarity may be “partial,” where the base pairing is less than 100%, or complementarity may be “complete” or “total,” implying perfect 100% antiparallel complementation between the two polynucleotides. By convention in the art, single-stranded nucleic acid molecules are written with their 5′ ends to the left, and their 3′ ends to the right.

The term “antigen,” as used herein, refers to any agent that is recognized by an antibody, while the term “immunogen” refers to any agent that can elicit an immunological response in a subject. The terms “antigen” and “immunogen” both encompass, but are not limited to, polypeptides. In most, but not all cases, antigens are also immunogens.

The term “allergen,” and grammatical variants thereof, as used herein, refer to antigens that are capable of inducing IgE-mediated responses, e.g., allergies. An allergen can be almost anything that acts as an antigen and stimulates an IgE-mediated allergic reaction. Common allergens can be found, for example, in pollens, molds, animal danders, insect (dust mites and cockroaches), foods and venom from insects such as bees, wasps and mosquitoes. Preferred allergens would be airborne allergens which include, but are not limited to, pollens, molds, animal danders and insect allergens (dust mites and cockroaches).

The terms “epitope” or “antigenic determinant” as used herein, refer to that portion of an antigen that determines the interaction with a particular antibody variable region, and thus imparts specificity to the antigen/antibody binding. A single antigen may have more than one epitope. An immunodominant epitope is an epitope on an antigen that is preferentially recognized by antibodies to the antigen. In some cases, where the antigen is a protein, the epitope can be “mapped,” and an “antigenic peptide” produced corresponding approximately to just those amino acids in the protein that are responsible for the antibody/antigen specificity. Such “antigenic peptides” find use in peptide immunotherapies.

The terms “disease,” “disorder” and “condition” are used interchangeably herein, and refer to any disruption of normal body function, or the appearance of any type of pathology. The etiological agent causing the disruption of normal physiology may or may not be known. Furthermore, although two patients may be diagnosed with the same disorder, the particular symptoms displayed by those individuals may or may not be identical.

The terms “type I allergic reaction,” “immediate hypersensitivity,” “atopic allergy,” “type-I hypersensitivity,” and the like, as used herein, refer to the physiological response that occurs when an antigen entering the body encounters mast cells or basophils which have been sensitized by IgE attached to its high-affinity receptor, FceRI on these cells. When an allergen reaches the sensitized mast cell or basophil, it cross-links surface-bound IgE, causing an increase in intracellular calcium (Ca²⁺) that triggers the release of pre-formed mediators, such as histamine and proteases, and newly synthesized, lipid-derived mediators such as leukotrienes and prostaglandins. These autocoids produce the clinical symptoms of allergy. In addition, cytokines, e.g., IL-4, TNF-alpha, are released from degranulating basophils and mast cells, and serve to augment the inflammatory response that accompanies an IgE reaction (see, e.g., Immunology, Fifth Edition, Roitt et al., eds., 1998, pp. 302-317). The specific manifestations of the hypersensitivity reaction in the sensitive or allergic subject depends on the site of the allergen exposure, the dose of allergen exposure, the reactivity of the organs in the subject (e.g., over-reactive lungs or nose) and the full panoply of the immune response to the allergen in that subject.

Symptoms and signs associated with type I hypersensitivity responses are extremely varied due to the wide range of tissues and organs that can be involved. These symptoms and signs can include, but are not limited to: itching of the skin, eyes, and throat, swelling and rashes of the skin (angioedema and urticaria/hives), hoarseness and difficulty breathing due to swelling of the vocal cord area, a persistent bumpy red rash that may occur anywhere on the body, shortness of breath and wheezing (from tightening of the muscles in the airways and plugging of the airways, i.e., bronchoconstriction) in addition to increased mucus and fluid production, chest tightness and pain due to construction of the airway muscles, nausea, vomiting diarrhea, dizziness and fainting from low blood pressure, a rapid or irregular heartbeat and even death as a result of airway and/or cardiac compromise.

Examples of disease states that result from allergic reactions, and demonstrating hypersensitivity symptoms and/or signs include, but are not limited to, allergic rhinitis, allergic conjunctivitis, atopic dermatitis, allergic [extrinsic] asthma, some cases of urticaria and angioedema, food allergy, and anaphylactic shock in which there is systemic generalized reactivity and loss of blood pressure that may be fatal.

The terms “anaphylaxis,” “anaphylactic response,” “anaphylactic reaction,” “anaphylactic shock,” and the like, as used interchangeably herein, describe the acute, often explosive, IgE-mediated systemic physiological reaction that occurs in a previously sensitized subject who receives the sensitizing antigen. Anaphylaxis occurs when the previously sensitizing antigen reaches the circulation. When the antigen reacts with IgE on basophils and mast cells, histamine, leukotrienes, and other inflammatory mediators are released. These mediators cause the smooth muscle contraction (responsible for wheezing and gastrointestinal symptoms) and vascular dilation (responsible for the low blood pressure) that characterize anaphylaxis. Vasodilation and escape of plasma into the tissues causes urticaria and angioedema and results in a decrease in effective plasma volume, which is the major cause of shock. Fluid escapes into the lung alveoli and may produce pulmonary edema. Obstructive angioedema of the upper airway may also occur. Arrhythmias and cardiogenic shock may develop if the reaction is prolonged. The term “anaphylactoid reaction” refers to a physiological response that displays characteristics of an anaphylactic response.

Symptoms of an anaphylactic reaction vary considerably among patients. Typically, in about 1 to 15 minutes (but rarely after as long as 2 hours), symptoms can include agitation and flushing, palpitations, paresthesias, pruritus, throbbing in the ears, coughing, sneezing, urticaria and angioedema, vasodilation, and difficulty breathing owing to laryngeal edema or bronchospasm. Nausea, vomiting, abdominal pain, and diarrhea are also sometimes observed. Shock may develop within another 1 or 2 minutes, and the patient may convulse, become incontinent, unresponsive, and succumb to cardiac arrest, massive angioedema, hypovolemia, severe hypotension and vasomotor collapse and primary cardiovascular collapse. Death may ensue at this point if the antagonist epinephrine is not immediately available. Mild forms of anaphylactic response result in various symptoms including generalized pruritus, urticaria, angioedema, mild wheezing, nausea and vomiting. Patients with the greatest risk of anaphylaxis are those who have reacted previously to a particular drug or antigen.

“Screening” as used herein refers to a procedure used to evaluate a subject for risk of type I allergic reaction or immediate hypersensitivity reaction when exposed to allergens in the presence of airborne pollutants. It is not required that the screening procedure be free of false positives or false negatives, as long as the screening procedure is useful and beneficial in determining which of those individuals within a group or population of individuals are at increased risk of an immediate hypersensitivity reaction. A screening procedure may be carried out for both prognostic and diagnostic purposes (i.e., prognostic methods and diagnostic methods).

“Prognostic method” refers to a method used to help predict, at least in part, the course of a disease. For example, a screening procedure may be carried out on a subject that has not previously been diagnosed with hypersensitivity to allergens, or does not show substantial allergic symptoms, when it is desired to obtain an indication of the future likelihood that the subject will be afflicted with type 1 allergic symptoms when exposed to allergen in the presence of airborne pollutants. In addition, a prognostic method may be carried out on a subject previously diagnosed with type 1 allergic reactions when it is desired to gain greater insight into how the disease will progress for that particular subject (e.g., the likelihood that a particular patient will respond favorably to a particular drug treatment). A prognostic method may also be used to determine whether a person will respond to a particular drug.

“Diagnostic method” as used herein refers to a screening procedure carried out on a subject that has previously been determined to be at risk for a type 1 hypersensitivity reaction to an allergen due to the presentation of symptoms or the results of another (typically different) screening test.

An enhanced or increased biological response to an allergen in the presence of airborne pollutants means that the level of IgE or histamine increases in the patient compared to the level of these compounds in the patient upon exposure to the allergen in the absence of the airborne pollutant. It is contemplated that the level of IgE or histamine may be 2 times greater in the presence of the airborne pollutant than in the absence of the pollutant. In another aspect the level is 3 times greater. In another aspect the level is 4 times greater in the presence of the airborne pollutant than in the absence of the pollutant.

“Functional polymorphism” as used herein refers to a change in the base pair sequence of a gene that produces a qualitative or quantitative change in the activity of the protein encoded by that gene. (e.g., a change in specificity of activity; a change in level of activity). The presence of a functional polymorphism indicates that the subject is at greater risk of developing a hypersensitivity reaction as compared to the general population. For example, the patient carrying the functional polymorphism may be particularly susceptible to chronic exposure to airborne pollutants that contribute to development of a allergic hypersensitivity or a type 1 allergic reaction. The term “functional polymorphism” includes mutations.

A “present” functional polymorphism as used herein (e.g., one that is indicative of or a risk factor for type 1 allergy) refers to the nucleic acid sequence corresponding to the functional polymorphism that is found less frequently in the general population relative to the population which exhibits type 1 allergic reaction. An “absent” functional polymorphism as used herein or “null” polymorphism means that the polymorphism results in the absence of a functional form of the protein.

“Mutation” as used herein sometimes refers to a functional polymorphism that occurs in less than one percent of the population, and is strongly correlated to the presence of a gene (i.e., the presence of such a mutation indicating a high risk of the subject being afflicted with a disease). However, “mutation” is also used herein to refer to a specific site and type of functional polymorphism, without reference to the degree of risk that particular mutation poses to an individual for a particular disease.

The term “primer” means a nucleic acid sequence used in the polymerase chain reaction (PCR) procedure to amplify the gene. The primer is preferably single stranded. The primer is preferably from 5 to 30 nucleic acids in length, more preferably from 10 to 25 nucleic acids in length. Preferably the primers for the GSTP1 gene are 5′-CCTGGTGGACATGGTGAATG-3′ (SEQ ID NO:7) and 5′TGCTCACACCATAGTTGGTGTAGATGA-3′ (SEQ ID NO:8). Preferably the primers for the GSTM1 gene are 5′-CTTGGAGGAACTCCCTGAAAAG-3′ (SEQ ID NO:1) and 5′-TGGAACCTCCATAACACGTGA-3′ (SEQ ID NO:2).

Fragments from the GSTM1 and GSTP1 genes can be used as hybridization probes. Optionally the length of the probes will be about 5 to 50 nucleotide bases, more preferably 10 to 30 nucleotide bases and more preferably 15 to 25 nucleotide bases. The hybridization probes may be derived from at least partially novel regions of the full length native nucleotide sequences. Hybridization probes may be labeled by a variety of labels, including radionucleotides such as ³²P or ³⁵S, or enzymatic labels such alkaline phosphatase coupled to the probe via avidin/biotin coupling systems. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3, 3′, 5, 5′-tetra-methylbenzidine (TMB), fluorescein, and its derivatives, dansyl, umbelliferone and the like or with horse radish peroxidase, alkaline phosphatase and the like.

The term “polypeptide”, in singular or plural, is used herein to refer to any peptide or protein comprising two or more amino acids joined to each other in a linear chain by peptide bonds. As used herein, the term refers to both short chains, which also commonly are referred to in the art as peptides, oligopeptides and oligomers, and to longer chains, commonly referred to in the art as proteins. Polypeptides, as defined herein, may contain amino acids other than the 20 naturally occurring amino acids, and may include modified amino acids. The modification can be anywhere within the polypeptide molecule, such as, for example, at the terminal amino acids, and may be due to natural processes, such as processing and other post-translational modifications, or may result from chemical and/or enzymatic modification techniques which are well known to the art. The known modifications include, without limitation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature, such as, for instance, Creighton, T. E., Proteins—Structure And Molecular Properties, 2nd Ed., W. H. Freeman and Company, New York (1993); Wold, F., Posttranslational Protein Modifications: Perspectives and Prospects,” in Posttranslational Covalent Modification of Proteins, Johnson, B. C., ed., Academic Press, New York (1983), pp. 1-12; Seifter et al., Analysis for protein modifications and nonprotein cofactors, Meth. Enzymol. 182:626-646 (1990), and Rattan et al., Ann. N.Y. Acad. Sci. 663:48-62 (1992).

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine. Accordingly, when glycosylation is desired, a polypeptide is expressed in a glycosylating host, generally eukaryotic host cells. Insect cells often carry out the same post-translational glycosylations as mammalian cells and, for this reason, insect cell expression systems have been developed to express efficiently mammalian proteins having native patterns of glycosylation.

It will be appreciated that polypeptides are not always entirely linear. For instance, polypeptides may be branched as a result of ubiquitination, and they may be circular, with or without branching, generally as a result of post-translational events, including natural processing and events brought about by human manipulation which do not occur naturally. Circular, branched and branched circular polypeptides may be synthesized by non-translation natural process and by entirely synthetic methods, as well. Such structures are within the scope of the polypeptides as defined herein.

Amino acids are represented by their common one- or three-letter codes, as is common practice in the art. Accordingly, the designations of the twenty naturally occurring amino acids are as follows: Alanine=Ala (A); Arginine=Arg (R); Aspartic Acid=Asp (D); Asparagine=Asn (N); Cysteine=Cys (C); Glutamic Acid=Glu (E); Glutamine=Gln (O); Glycine=Gly (G); Histidine=His (H); Isoleucine=Ile (I); Leucine=Leu (L); Lysine=Lys (K); Methionine=Met (M); Phenylalanine=Phe (F); Proline—Pro (P); Serine=Ser (S); Threonine=Thr (T); Tryptophan=Trp (W); Tyrosine=Tyr (Y); Valine=Val (V). The polypeptides herein may include all L-amino acids, all D-amino acids or a mixture thereof. The polypeptides comprised entirely of D-amino acids may be advantageous in that they are expected to be resistant to proteases naturally found within the human body, and may have longer half-lives.

The term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have at least one amino acid deleted in a particular region of the molecule.

The terms “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-associated virus (AAV)), DNA encapsulated in liposomes, DNA complexed with polylysine, complexed with synthetic polycationic molecules, conjugated with transferrin, complexed with compounds such as polyethylene glycol (PEG) to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A “host cell” includes an individual cell or cell culture which can be or has been a recipient of any vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a nucleic acid of the present invention.

The term “promoter” means a nucleotide sequence that, when operably linked to a DNA sequence of interest, promotes transcription of that DNA sequence.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accord with conventional practice.

The term “IgE-mediated biological response” is used to refer to a condition or disease which is characterized by signal transduction through an IgE receptor, including the high-affinity IgE receptor, Fcε.R1, and the low-affinity IgE receptor FcεR11. The definition includes, without limitation, conditions associated with anaphylactic hypersensitivity and atopic allergies, such as, for example, asthma, allergic rhinitis, atopic dermatitis, food allergies, chronic urticaria and angioedema, as well as the serious physiological condition of anaphylactic shock.

The terms “treat” or “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain a desired effect or level of agent(s) for an extended period of time.

“Intermittent” administration is treatment that is not consecutively done without interruption, but rather is periodic in nature.

Administration “in combination with” one or more further therapeutic agents includes simultaneous (concurrent) and consecutive administration in any order.

An “effective amount” is an amount sufficient to effect beneficial or desired therapeutic (including preventative) results. An effective amount can be administered in one or more administrations.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™., polyethylene glycol (PEG), and PLURONICS™.

### II. Detailed Description

Allergic Conditions

Immediate hypersensitivity or Type I allergy is manifested in a broad array of conditions and associated symptoms, which may be mild, chronic, acute and/or life threatening. These various pathologies include, for example, allergic asthma, allergic rhinitis, atopic dermatitis, severe food allergies, chronic urticaria and angioedema, as well as the serious physiological condition of anaphylactic shock. A wide variety of antigens are known to act as allergens, and exposure to these allergens results in the allergic pathology. Common allergens include, but are not limited to, bee stings, penicillin, various food allergies, pollens, animal detritus (especially house dust mite, cat, dog and cockroach), and fungal allergens. The most severe responses to allergens can result in airway constriction and anaphylactic shock, both of which are potentially fatal conditions.

It is now clear that severity of allergic airway diseases such as allergic rhinitis and asthma is a consequence of the interplay between genes and environment. Underpinning these diseases are the formation of a Th2 cytokine environment and the production of allergen-specific IgE antibodies.

In conjunction with allergen, diesel exhaust particles and environmental tobacco smoke can act as an adjuvant to enhance IgE antibody responses, T-helper 2 (Th2) cytokine production, and histamine release in vivo. As with other inhaled pollutants, diesel exhaust particles are thought to exert major effects through production of reactive oxygen species.

Most studies postulate that the environmental tobacco smoke effect is primarily mechanical as a direct irritant causing a gross inflammatory process which leads to airway damage and a subsequent impairment of airway caliber or bronchial responsiveness.

It has now been determined that in humans, environmental tobacco smoke can work through an additional mechanism of adjuvancy, i.e., the interaction with allergen to alter the immune system and enhance allergic responses. As noted before, environmental tobacco smoke exposure in children has been associated with increased serum IgE and skin test reactivity (presumably due to heightened histamine release). However, other studies have failed to observe any association. (Ownby et al., J Allergy Clin Immunol 1988; 82:634-8). We and others have shown that ETS exposure exerts an adjuvant effect in mice characterized by an increase in antigen-specific IgE, elevated Th2 responses, and influx of eosinophils into the lungs (Rumold et al., J Immunol 2001; 167:4765-70; Seymour et al.,. J Immunol 1997; 159:6169-75). These studies have also shown that ETS can augment primary sensitization to an innocuous protein.

Several endogenous small molecules and proteins are involved in airway antioxidant defenses (Gilliland et al., Environ Health Perspect 1999: 107 (suppl): 403-07). Glutathione-S-transferases (GSTs) are a large family of proteins that participate in antioxidant defenses through several mechanisms including reactive oxygen species metabolism and detoxification of xenobiotics. GSTM1, GSTT1, and GSTP1 genotypes are expressed in the respiratory tract, are involved in detoxification of chemicals, and have common functional variant alleles (Talalay et al., Proc Natl Acad Sci USA 1995; 92: 8965-69). These variant alleles result in either total absence or a substantial change in enzyme activity. Furthermore, three members of this superfamily GSTM1, GSTT1, and GSTP1 with common genetic variants are thought to affect asthma (Fryer et al., Am J. Respir Cell Mol. Biol. 2002 161: 1437-42; Gilliland et al., Am J. Respir Crit. Care Med 2002: 166 457-63; Mapp et al., J. Allergy Clim Immunol. 2002 109: 867-72).

Susceptibility to an adverse health effect of diesel exhaust particles, a model oxidant pollutant, can be controlled by functional variation in natural antioxidant defenses. Several polymorphic genes including those for GSTs have been associated with atopy (allergy, asthma, and atopic dermatitis). It has now been determined that the GSTM1 and GSTP1 genotypes play an important part in susceptibility to the adjuvant effects of oxidant pollutants such as diesel exhaust particles and environmental tobacco smoke.

GSTM1 is present in lung and nasal tissue although its expression is highest in the liver (Hayes et al., Crit Rev Biochem Mol. Biol. 199530:445-600). Different members of the GST family use distinct but overlapping substrates. It is notable that GSTM1 is involved mainly with detoxification of oxy-polyaromatic hydrocarbons. GSTP1 detoxifies lipid peroxidation products and DNA oxidation products.

The importance of these results is heightened by the high frequency of polymorphisms of these genes in most populations. For example, the null allele variant of GSTM1 occurs in about 50% of individuals (To-Figueras et al., Carcinogenesis 1997; 18:1529-33). GSTP1 I/I genotype frequency is roughly 40%. Because the genes are on different chromosomes, they assort independently. On the basis of this information, it is estimated that 15-20% of the general population are at the highest risk for a large enhancement of allergic responses due to exposure to diesel exhaust particles. Among individuals who are allergic, the proportion of the population at risk for diesel exhaust particles enhancement could be larger than the proportion among the non-allergic population.

Results of epidemiological studies (Fryer et al., Am J. Respir Cell Mol. Biol. 2002 161: 1437-42; Mapp et al., J. Allergy Clim Immunol. 2002 109: 867-72; Piirila et al., Pharmacogenetics 2001: 11:437-45) have shown that the GSTP1 and GSTM1 polymorphisms are associated with airway hyper-responsiveness and asthma, especially in those whose asthma is related to xenobiotic exposure. Furthermore, the frequency of the GSTP1 V105/V105 genotype is reduced in patients who are atopic compared with those who are not (Fryer et al., Am J. Respir Cell Mol. Biol. 2002 161: 1437-42). Airway inflammation is thought to result in formation of reactive oxygen species and small molecular and enzymatic antioxidants can mitigate the formation and effects of reactive oxygen species. (Gilliland et al., Environ Health Perspect 1999: 107 (suppl): 403-07). GSTs might also affect synthesis of eiconosoids such as leucotrienes that modulate allergic responses.

To assess the extent to which the reported variability reported in responses to diesel exhaust particles is explained by these polymorphisms, the joint GSTM1 null and GSTP1 I/I genotype, which show the largest enhancement of allergic responses from diesel exhaust particles was examined.

It has now been found that an additional role for GSTs is that members of the GST family can play a key part in controlling the response to diesel exhaust particles and environmental smoke by detoxifying reactive oxygen species derived from diesel exhaust. Studies in mice (Diaz-Sanchez, D. Immunology 2000, 101:1-13) have shown that an antioxidant will block production of interleukin 4 and IgE that is enhanced by diesel exhaust particles. Many in-vitro studies also suggest that the effect of diesel exhaust particles on interleukin 4 is due to generation of oxidative stress. It has previously been shown that the earliest detectable source of interleukin 4 after nasal challenge with diesel exhaust particles plus allergen derives from CD117 positive cells. (Wang et al., Clin Immunol 1999 90:47-54). The failure to find significant differences in interleukin 4 and interferon-γ induction related to allergen exposure in the presence of diesel exhaust particles is probably due to a type 2 error.

It has also been found that a small increase in interleukin 4 and a small reduction in interferon-γ result in a large change in IgE. Formation of allergic antibodies is a complex process that involves many cytokines and processes. Enhancement of interleukin 4 by diesel exhaust particles probably leads to a cascade effect resulting in promotion of a Th-2 environment including further production of interleukin 4, interleukin 13, and interleukin 6. In addition, diesel exhaust particles can increase antigen presentation and T-cell responses. The combination of these factors leads to a more robust increase in IgE production than does each factor alone.

Particles in diesel exhaust have been used as a model particulate pollutant. Diesel exhaust particles make up to 40% of the PM, found in the air in the Los Angeles basin. Results of studies in people and animals (Wang et al., Clin Immunol 1999 90:47-54; Manchester et al., Determination of the elemental carbon, organic carbon and source contributions to atmospheric particles during the southern California Children's Health Study, Part B, 1995. Sacramento Calif.: California Resources Board 2001: 1-38) have shown that diesel exhaust particles can participate in both starting and enhancing allergic immune responses.

In the study of diesel exhaust particles, participants were exposed to an amount estimated to be equivalent to 40 hours of exposure of people living in Los Angeles. This amount of diesel exhaust particles can act as an adjuvant when given with allergen to augment IgE, Th-2 cytokine, and chemokine production while increasing symptom severity and histamine release (Diaz-Sanchez et al., J. Immunol. 1997 158:2406-13; Diaz-Sanchez et al., J. Allergy Clin Immunol. 2001 106;1140-46, Diaz-Sanchez et al., Clin Immunol 2000 97:140-45). Evidence for involvement of reactive oxygen species generation in diesel exhaust particles' health effects has come from both human and animal exposure models. In mice models of asthma, diesel exhaust particles can increase cytochrome P450 reductase activity in the lung while decreasing oxygen scavenging ability (Sagai et al., Involvements of superoxide and nitric oxide on asthma-like features induced by diesel exhaust particles in mice Basel: S. Karger, 1998). Pretreatment of these mice with antioxidants will decrease eosinophilia induced by diesel exhaust particles, mucus hyper-secretion, airway hyper-responsiveness, and IgE responses to bystander antigen (Sagai et al., Involvements ofsuperoxide and nitric oxide on asthma-like features induced by diesel exhaust particles in mice Basel: S. Karger, 1998; Whitekus et al., J. Immunol 2002 168:2560-67). Additionally, Nightingale and colleagues (Am J. Respir Crit Care Med 2000 162 161-66) showed that exposure of healthy volunteers to resuspended diesel exhaust particles (200 μg/m³) in an inhalation chamber results in an increase in sputum inflammatory cells along with an increase in exhaled carbon monoxide concentrations, an indicator of oxidant stress.

Diesel exhaust particles consist of a carbon core surrounded by chemicals including quinones and polyaromatic hydrocarbons, which can be metabolised to produce oxy-polyaromatic hydrocarbons (Williams et al., Fuel 1986 65:1150-58). These chemicals can induce reactive oxygen species leading to activation of intracellular signaling pathways and induction of gene transcription (Nel et al., Curr Opin Pulm Med 20017:20-26). Generation of hydroxyl radicals induces a decrease in glutathione (GSH) concentrations, which in turn will increase transcription of GST genes. The GST member with the most pronounced effect on diesel exhaust particles sensitivity is GSTM1, a class μ GST isoenzyme.

GSTM1 might be involved in restricting initial generation of the reactive oxygen species response by diesel exhaust particles chemicals whereas GSTP1 plays a part in a later stage when inflammation and oxidant damage is taking place.

Inhalation challenge studies are useful models and have been used extensively to study the potential of environmental agents to change the immune response under controlled conditions. This system has been used to investigate airway responses to diesel exhaust particles, ozone, second-hand smoke, sulphur dioxide, and nitrous oxide, among others (Bascom et al., Am. Rev. Respir. Dis. 1990 142:594-601; Bascom, R. Pharmacogenetics 1991 1:102-06; Trenga et al., Occup Environ Med 1999 56:544-47; Strand et al., Am. J. Respir. Crit Care Med 1997 155:881-87). A common feature of these challenges is observation of both interindividual variability and intraindividual consistency such that a non-sensitive individual will always have a weak or small response to the pollutant.

In view of the substantial effects of GSTM1 and GSTP1 variants on enhancement of allergic responses by diesel exhaust particles and environmental tobacco smoke, our results suggest that these genes have an important role in modification of the airway response to diesel exhaust particles and environmental tobacco smoke. These results, therefore, have obvious clinical and public-health relevance especially for sensitized individuals living in urban environments. Because the GSTM1 and GSTP1 variants we investigated are common, the number of susceptible individuals with symptomatic allergic airway disease in the setting of airborne pollutants would be expected to be large. Furthermore, small molecule antioxidants and dietary intake might contribute to antioxidant defenses directly or by increasing expression of antioxidant genes.

It would be useful for devising treatment regimens to identify those patients who are susceptible to increased allergic airway disease in the presence of airborne pollutants.

There is evidence to suggest that the results also apply to the lower airway. Diesel exhaust particles can enhance airway hyper-reactivity in mice. This hyper-reactivity is associated with increased production of reactive oxygen species in the alveolar spaces (Hiura et al., J. Immunol. 2000 165:2703-11). In addition, exposure to diesel exhaust particles will change intracellular glutathione concentrations in alveolar macrophages and lymphocytes (Al-Humadi et al., Environ Health Perspect 2002 110:349-53). Furthermore, the GSTP1 gene product might provide more than 90% of the glutathione-S-transferase activity in the lung (Fryer et al., Biochim Biophys Acta 1986 883:448-53). It is contemplated that lower airway and upper airway effects of diesel exhaust particles are mostly modulated by GSTP1.

This invention is directed to the detection of genetic polymorphisms. The polymorphism may be identified by any method known to one of ordinary skill in the art which identifies the presence or absence of the particular allele or marker, including, for example, direct sequencing single-strand conformation polymorphism analysis (SSCP), base excision sequence scanning (BESS), RFLP analysis, heteroduplex analysis, denaturing gradient gel electrophoresis, allelic PCR, temperature gradient electrophoresis, ligase chain reaction, direct sequencing, minisequencing, nucleic acid hybridization, and micro-array-type detection of the GSTM1 or GSTP1 genes. Yet another technique includes an Invader Assay which includes isothermic amplification that relies on a catalytic release of fluorescence. All of these techniques are intended to be within the scope of the invention. A brief description of these techniques follows.

Isolation and Amplification of Nucleic Acid

Samples of patient, test subject, or family member genomic DNA are isolated from any convenient biological sample including but not limited to saliva, buccal cells, hair roots, blood, cord blood, amniotic fluid, interstitial fluid, peritoneal fluid, chorionic villus, and any other suitable cell or tissue sample with intact interphase nuclei or metaphase cells. The cells can be obtained from solid tissue as from a fresh or preserved organ or from a tissue sample or biopsy. The sample can contain compounds which are not naturally intermixed with the biological material such as preservatives, anticoagulants, buffers, fixatives, nutrients, antibiotics, or the like.

Methods for isolation of genomic DNA from these various sources are described in, for example, Kirby, DNA Fingerprinting, An Introduction, W.H. Freeman & Co. New York (1992). Genomic DNA can also be isolated from cultured primary or secondary cell cultures or from transformed cell lines derived from any of the aforementioned tissue samples.

Samples of patient, test subject or family member RNA can also be used. RNA can be isolated from tissues expressing the GSTM1 or GSTP1 gene as described in Sambrook et al., Molecular Cloning A Laboratory Manual 3rd Ed. Cold Spring Harbor Press New York 2001. RNA can be total cellular RNA, mRNA, poly A+ RNA, or any combination thereof. For best results, the RNA is purified, but can also be unpurified cytoplasmic RNA. RNA can be reverse transcribed to form DNA which is then used as the amplification template, such that the PCR indirectly amplifies a specific population of RNA transcripts. See, e.g., Sambrook, supra, and Berg et al., Hum. Genet. 85:655-658 (1990).

PCR Amplification

The most common means for amplification is polymerase chain reaction (PCR), as described in U.S. Pat. Nos. 4,683,195, 4,683,202, 4,965,188 each of which is hereby incorporated by reference. If PCR is used to amplify the target regions in blood cells, heparinized whole blood should be drawn in a sealed vacuum tube kept separated from other samples and handled with clean gloves. For best results, blood should be processed immediately after collection; if this is impossible, it should be kept in a sealed container at 4° C. until use. Cells in other physiological fluids may also be assayed. When using any of these fluids, the cells in the fluid should be separated from the fluid component by centrifugation.

Tissues should be roughly minced using a sterile, disposable scalpel and a sterile needle (or two scalpels) in a 5 mm Petri dish. Procedures for removing paraffin from tissue sections are described in a variety of specialized handbooks well known to those skilled in the art.

To amplify a target nucleic acid sequence in a sample by PCR, the sequence must be accessible to the components of the amplification system. One method of isolating target DNA is crude extraction which is useful for relatively large samples. Briefly, mononuclear cells from samples of blood, buccal cells, or the like are isolated and centrifuged at 2000 g on the day they are received. The pellets are stored frozen at −20° C. until used. DNA is extracted using a PUREGENE® DNA isolation kit (Cat#D-5000, GENTRA, Minneapolis, Minn.). The GSTM1 and GSTP1 genotypes can be determined using real-time polymerase chain reaction using a TaqMan 7700 (Applied Biosystems, Foster City Calif.).

The pellets are resuspended in lysis solution from the PUREGENE® DNA isolation kit containing 100 μg/ml of proteinase K. After incubating at 55° C. overnight. DNA extraction is performed according to manufacturers recommendations. The DNA samples are resuspended in aqueous solution and stored at −20° C.

The presence or absence of a fluorescent amplification signal may be used as an indication of whether the GSTM1 or GSTP1 alleles are present or absent in a particular genomic DNA sample. Samples showing no signal or late cycle number for start amplification can be repeated and further analyzed with primers and probes for the actin gene to verify the presence of amplifiable DNA.

Analysis of the single nucleotide polymorphism at codon 105 in the GSTP 1 gene can be performed using allele-specific probes.

When extracting DNA from tissues, the amount of the above mentioned buffer with proteinase K may vary according to the size of the tissue sample. The extract may be incubated for 4-10 hrs at 50° C.-60° C. and then at 95° C. for 10 minutes to inactivate the proteinase. During longer incubations, fresh proteinase K may be added after about 4 hr at the original concentration.

When the sample contains a large number of cells, extraction may be accomplished by methods as described in Higuchi, “Simple and Rapid Preparation of Samples for PCR”, in PCR Technology, p. 31-43 Ehrlich, H. A. (ed.), Stockton Press, New York. PCR can be employed to amplify target regions in a large number of cells derived from bone marrow and peripheral blood cultures.

A relatively easy procedure for extracting DNA for PCR is a salting out procedure adapted from the method described by Miller et al., Nucleic Acids Res. 16:1215 (1988), which is incorporated herein by reference. Buffy coats of nucleated cells are resuspended in 3 ml of lysis buffer (10 mM Tris-HCl, 400 mM NaCl, 2 mM Na₂ EDTA, pH 8.2). Fifty μl of a 20 mg/ml solution of proteinase K and 200 μl of a 20% SDS solution are added to the cells and then incubated at 37° C. overnight. Following adequate digestion, one ml of a 6M NaCl solution is added to the sample and vigorously mixed. The resulting solution is centrifuged for 15 minutes at 2500 rpm. The pellet contains the precipitated cellular proteins, while the supernatant contains the DNA. The supernatant is removed to a 15 ml tube that contains 2 volumes of absolute ethanol. The contents of the tube are mixed gently until the water and the alcohol phases have mixed and a white DNA precipitate has formed. The DNA precipitate is placed in distilled water and dissolved.

Kits for the extraction of high-molecular weight DNA for PCR include PUREGENE® DNA Isolation kit (D-5000) GENTRA, a Genomic Isolation Kit A.S.A.P.® (Boehringer Mannheim, Indianapolis, Ind.), Genomic DNA Isolation System (GIBCO BRL, Gaithersburg, Md.), ELU-QUIK® DNA Purification Kit (Schleicher & Schuell, Keene, N.H.), DNA Extraction Kit (Stratagene, LaJolla, Calif.), TURBOGEN® Isolation Kit (Invitrogen, San Diego, Calif.), and the like. Use of these kits according to the manufacturer's instructions is generally acceptable for purification of DNA prior to practicing the methods of the present invention.

The concentration and purity of the extracted DNA can be determined by spectrophotometric analysis of the absorbance of a diluted aliquot at 260 nm and 280 nm.

After extraction of the DNA, PCR amplification may proceed. The first step of each cycle of the PCR involves the separation of the nucleic acid duplex formed by the primer extension. Once the strands are separated, the next step in PCR involves hybridizing the separated strands with primers that flank the target sequence. The primers are then extended to form complementary copies of the target strands. For successful PCR amplification, the primers are designed so that the position at which each primer hybridizes along a duplex sequence is such that an extension product synthesized from one primer, when separated from the template (complement), serves as a template for the extension of the other primer. The cycle of denaturation, hybridization, and extension is repeated as many times as necessary to obtain the desired amount of amplified nucleic acid.

In one embodiment of PCR amplification, strand separation is achieved by heating the reaction to a sufficiently high temperature for a sufficient time to cause the denaturation of the duplex but not to cause an irreversible denaturation of the polymerase (see U.S. Pat. No. 4,965,188, incorporated herein by reference). Typical heat denaturation involves temperatures ranging from about 80° C. to 105° C. for times ranging from seconds to minutes. Strand separation, however, can be accomplished by any suitable denaturing method including physical, chemical, or enzymatic means. Strand separation may be induced by a helicase, for example, or an enzyme capable of exhibiting helicase activity. For example, the enzyme RecA has helicase activity in the presence of ATP. The reaction conditions suitable for strand separation by helicases are known in the art (see Kuhn et al., 1979, CSH-Quantitative Biology, 43:63-67; and Radding, 1982, Ann. Rev. Genetics 16:405-437).

Template-dependent extension of primers in PCR is catalyzed by a polymerizing agent in the presence of adequate amounts of four deoxyribonucleotide triphosphates (typically dATP, dGTP, dCTP, and dTTP) in a reaction medium comprised of the appropriate salts, metal cations, and pH buffering systems. Suitable polymerizing agents are enzymes known to catalyze template-dependent DNA synthesis. In some cases, the target regions may encode at least a portion of a protein expressed by the cell. In this instance, mRNA may be used for amplification of the target region. Alternatively, PCR can be used to generate a cDNA library from RNA for further amplification, the initial template for primer extension is RNA. Polymerizing agents suitable for synthesizing a complementary, copy-DNA (cDNA) sequence from the RNA template are reverse transcriptase (RT), such as avian myeloblastosis virus RT, Moloney murine leukemia virus RT, or Thermus thermophilus (Tth) DNA polymerase, a thermostable DNA polymerase with reverse transcriptase activity marketed by Perkin Elmer Cetus, Inc. Typically, the genomic RNA template is heat degraded during the first denaturation step after the initial reverse transcription step leaving only DNA template. Suitable polymerases for use with a DNA template include, for example, E. coli DNA polymerase I or its Klenow fragment, T4 DNA polymerase, Tth polymerase, and Taq polymerase, a heat-stable DNA polymerase isolated from Thermus aquaticus and commercially available from Perkin Elmer Cetus, Inc. The latter enzyme is widely used in the amplification and sequencing of nucleic acids. The reaction conditions for using Taq polymerase are known in the art.

Allele Specific PCR

Allele-specific PCR differentiates between target regions differing in the presence of absence of a variation or polymorphism. PCR amplification primers are chosen which bind only to certain alleles of the target sequence. This method is described by Gibbs, Nucleic Acid Res. 17:2437-2448 (1989).

Allele Specific Oligonucleotide Screening Methods

Further diagnostic screening methods employ the allele-specific oligonucleotide (ASO) screening methods, as described by Saiki et al., Nature 324:163 166 (1986). Oligonucleotides with one or more base pair mismatches are generated for any particular allele. ASO screening methods detect mismatches between variant target genomic or PCR amplified DNA and non-mutant oligonucleotides, showing decreased binding of the oligonucleotide relative to a mutant oligonucleotide. Oligonucleotide probes can be designed that under low stringency will bind to both polymorphic forms of the allele, but which at high stringency, bind to the allele to which they correspond. Alternatively, stringency conditions can be devised in which an essentially binary response is obtained, i.e., an ASO corresponding to a variant form of the target gene will hybridize to that allele, and not to the wildtype allele.

Ligase Mediated Allele Detection Method

Target regions of a test subject's DNA can be compared with target regions in unaffected and affected family members by ligase-mediated allele detection. Ligase may also be used to detect point mutations in the ligation amplification reaction described in Wu and Wallace., Genomics 4:560-569 (1989). The ligation amplification reaction (LAR) utilizes amplification of specific DNA sequence using sequential rounds of template dependent ligation as described in Wu, supra, and Barany, Proc. Nat. Acad. Sci. 88:189-193 (1990).

Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. DNA molecules melt in segments, termed melting domains, under conditions of increased temperature or denaturation. Each melting domain melts cooperatively at a distinct, base-specific melting temperature (TM). Melting domains are at least 20 base pairs in length, and may be up to several hundred base pairs in length.

Differentiation between alleles based on sequence specific melting domain differences can be assessed using polyacrylamide gel electrophoresis, as described in Myers et al., Chapter 7 of Erlich, ed., PCR Technology, W.H. Freeman and Co., New York (1989).

Generally, a target region to be analyzed by denaturing gradient gel electrophoresis is amplified using PCR primers flanking the target region. The amplified PCR product is applied to a polyacrylamide gel with a linear denaturing gradient as described in Myers et al., Meth. Enzymol. 155:501-527 (1986), and Myers et al., in Genomic Analysis, A Practical Approach, K. Davies Ed. IRL Press Limited, Oxford, pp. 95-139 (1988). The electrophoresis system is maintained at a temperature slightly below the Tm of the melting domains of the target sequences.

In an alternative method of denaturing gradient gel electrophoresis, the target sequences may be initially attached to a stretch of GC nucleotides, termed a GC clamp, as described in Chapter 7 of Erlich, supra. Preferably, at least 80% of the nucleotides in the GC clamp are either guanine or cytosine. Preferably, the GC clamp is at least 30 bases long. This method is particularly suited to target sequences with high Tm's.

Generally, the target region is amplified by the polymerase chain reaction as described above. One of the oligonucleotide PCR primers carries at its 5′ end, the GC clamp region, at least 30 bases of the GC rich sequence, which is incorporated into the 5′ end of the target region during amplification. The resulting amplified target region is run on an electrophoresis gel under denaturing gradient conditions as described above. DNA fragments differing by a single base change will migrate through the gel to different positions, which may be visualized by ethidium bromide staining.

Temperature Gradient Gel Electrophoresis.

Temperature gradient gel electrophoresis (TGGE) is based on the same underlying principles as denaturing gradient gel electrophoresis, except the denaturing gradient is produced by differences in temperature instead of differences in the concentration of a chemical denaturant. Standard TGGE utilizes an electrophoresis apparatus with a temperature gradient running along the electrophoresis path. As samples migrate through a gel with a uniform concentration of a chemical denaturant, they encounter increasing temperatures. An alternative method of TGGE, temporal temperature gradient gel electrophoresis (TTGE or tTGGE) uses a steadily increasing temperature of the entire electrophoresis gel to achieve the same result. As the samples migrate through the gel the temperature of the entire gel increases, leading the samples to encounter increasing temperature as they migrate through the gel. Preparation of samples, including PCR amplification with incorporation of a GC clamp, and visualization of products are the same as for denaturing gradient gel electrophoresis. (U.S. Patent Application No. 20040253594)

Single-Strand Conformation Polymorphism Analysis.

Target sequences or alleles at the GSTM1 or GSTP1 locus can be differentiated using single-strand conformation polymorphism analysis, which identifies base differences by alteration in electrophoretic migration of single stranded PCR products, as described in Orita et al., Proc. Nat. Acad. Sci. 86:2766-2770 (1989). Amplified PCR products can be generated as described above, and heated or otherwise denatured, to form single stranded amplification products. Single-stranded nucleic acids may refold or form secondary structures which are partially dependent on the base sequence. Thus, electrophoretic mobility of single-stranded amplification products can detect base-sequence difference between alleles or target sequences.

Chemical or Enzymatic Cleavage of Mismatches.

Differences between target sequences can also be detected by differential chemical cleavage of mismatched base pairs, as described in Grompe et al., Am. J. Hum. Genet. 48:212-222 (1991). In another method, differences between target sequences can be detected by enzymatic cleavage of mismatched base pairs, as described in Nelson et al., Nature Genetics 4:11-18 (1993). Briefly, genetic material from a patient and an affected family member may be used to generate mismatch free heterohybrid DNA duplexes. As used herein, “heterohybrid” means a DNA duplex strand comprising one strand of DNA from one person, usually the patient, and a second DNA strand from another person, usually an affected or unaffected family member. Positive selection for heterohybrids free of mismatches allows determination of small insertions, deletions or other polymorphisms.

Non-PCR Based DNA Diagnostics.

The identification of a DNA sequence linked to GSTM1 or GSTP1 can be made without an amplification step, based on polymorphisms including restriction fragment length polymorphisms in a patient and a family member. Hybridization probes are generally oligonucleotides which bind through complementary base pairing to all or part of a target nucleic acid. Probes typically bind target sequences lacking complete complementarity with the probe sequence depending on the stringency of the hybridization conditions. The probes are preferably labeled directly or indirectly, such that by assaying for the presence or absence of the probe, one can detect the presence or absence of the target sequence. Direct labeling methods include radioisotope labeling, such as with ³²P or ³⁵S. Indirect labeling methods include fluorescent tags, biotin complexes which may be bound to avidin or streptavidin, or peptide or protein tags. Visual detection methods include photoluminescents, Texas red, rhodamine and its derivatives, red leuco dye and 3, 3′, 5,5′-tetra-methylbenzidine (TMB), fluorescein, and its derivatives, dansyl, umbelliferone and the like or with horse radish peroxidase, alkaline phosphatase and the like.

One or more additional restriction enzymes and/or probes and/or primers can be used. Additional enzymes, constructed probes, and primers can be determined by routine experimentation by those of ordinary skill in the art and are intended to be within the scope of the invention.

Although the methods described herein may be in terms of the use of a single restriction enzyme and a single set of primers, the methods are not so limited. One or more additional restriction enzymes and/or probes and/or primers can be used, if desired. Additional enzymes, constructed probes and primers can be determined through routine experimentation, combined with the teachings provided and incorporated herein.

The reagents suitable for applying the methods of the invention may be packaged into convenient kits. The kits provide the necessary materials, packaged into suitable containers. At a minimum, the kit contains a reagent that identifies a polymorphism in the selected gene that is associated with a trait. Preferably, the reagent is a PCR set (a set of primers, DNA polymerase and 4 nucleoside triphosphates) that hybridize with the gene or a fragment thereof. Preferably, the PCR set is included in the kit. Preferably, the kit further comprises additional means, such as reagents, for detecting or measuring the detectable entity or providing a control. Other reagents used for hybridization, prehybridization, DNA extraction, visualization etc. may also be included, if desired.

A sample of genomic DNA may be evaluated by reference to one or more controls to determine if a polymorphism in the gene is present. Preferably, PCR analysis is performed with respect to the gene, and the results are compared with a control. The control is the result of a PCR analysis of the gene of a subject where the polymorphism of the gene is known. Similarly, the genotype of an subject may be determined by obtaining a sample of its mRNA or genomic DNA, conducting PCR analysis of the gene in the DNA, and comparing the results with a control. Again, the control is the result of PCR analysis of the same gene of a different subject. The results genetically type the subject by specifying the polymorphism in its selected gene. Finally, genetic differences among patients can be detected by obtaining samples of the mRNA or genomic DNA from at least two patients, identifying the presence or absence of a polymorphism in the gene, and comparing the results.

Preparation of the GSTM1 or GSTP1 Proteins

The GSTM1 and GSTP1 105 val proteins can be prepared by well known methods of recombinant DNA technology or traditional chemical extraction or synthesis. If the polypeptides are produced by recombinant host cells, cDNA encoding the desired polypeptide of the present invention is inserted into a replicable vector for cloning and expression. As discussed, the nucleotide and amino acid sequences for the GSTM1 and GSTP1 105val proteins are well known in the art and are readily available.

Suitable vectors are prepared using standard techniques of recombinant DNA technology, and are, for example, described in “Molecular Cloning: A Laboratory Manual”, 2nd edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); Handbook of Experimental Immunology, 4th edition (D. M. Weir & C. C. Blackwell, eds., Blackwell Science Inc., 1987); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); and “Current Protocols in Immunology” (J. E. Coligan et al., eds., 1991). Isolated plasmids and DNA fragments are cleaved, tailored, and ligated together in a specific order to generate the desired vectors. After ligation, the vector containing the gene to be expressed is transformed into a suitable host cell.

Host cells can be any eukaryotic or prokaryotic hosts known for expression of heterologous proteins. Accordingly, the polypeptides of the present invention can be expressed in eukaryotic hosts, such as eukaryotic microbes (yeast) or cells isolated from multicellular organisms (mammalian cell cultures), plants and insect cells. Examples of mammalian cell lines suitable for the expression of heterologous polypeptides include monkey kidney CV1 cell line transformed by SV40 (COS-7, ATCC CRL 1651); human embryonic kidney cell line 293S (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CV1-76, ATCC CCL 70); African green monkey cells (VERO-76, ATCC CRL-1587); human cervical carcinoma cells (HELA, ATCC CCL 2); canine kidney cells (MDCK, ATCC CCL 34); human lung cells (W138, ATCC CCL 75); and human liver cells (Hep G2, HB 8065).

Eukaryotic expression systems employing insect cell hosts may rely on either plasmid or baculoviral expression systems. The typical insect host cells are derived from the fall army worm (Spodoptera frugiperda). For expression of a foreign protein these cells are infected with a recombinant form of the baculovirus Autographa californica nuclear polyhedrosis virus which has the gene of interest expressed under the control of the viral polyhedrin promoter. Other insects infected by this virus include a cell line known commercially as “High 5” (Invitrogen) which is derived from the cabbage looper (Trichoplusia ni). Another baculovirus sometimes used is the Bombyx mori nuclear polyhedorsis virus which infect the silk worm (Bombyx mori). Numerous baculovirus expression systems are commercially available, for example, from Invitrogen (Bac-N-Blue™.), Clontech (BacPAK™. Baculovirus Expression System), Life Technologies (BAC-TO-BAC™.), Novagen (Bac Vector System™.), Pharmingen and Quantum Biotechnologies). Another insect cell host is common fruit fly, Drosophila melanogaster, for which a transient or stable plasmid based transfection kit is offered commercially by Invitrogen (The DES™. System).

Saccharomyces cerevisiae is the most commonly used among lower eukaryotic hosts. However, a number of other genera, species, and strains are also available and useful herein, such as Pichia pastoris (EP 183,070; Sreekrishna et al., J. Basic Microbiol. 28:165-278 (1988)). Yeast expression systems are commercially available, and can be purchased, for example, from Invitrogen (San Diego, Calif.). Other yeasts suitable for bi-functional protein expression include, without limitation, Kluyveromyces hosts (U.S. Pat. No. 4,943,529), e.g. Kluyveromyces lactis; Schizosaccharomyces pombe (Beach and Nurse, Nature 290:140 (1981); Aspergillus hosts, e.g. A. niger (Kelly and Hynes, EMBO J. 4:475-479 (1985])) and A. nidulans (Ballance et al., Biochem. Biophys. Res. Commun. 112:284-289 (1983)), and Hansenula hosts, e.g. Hansenula polymorpha. Yeasts rapidly growth on inexpensive (minimal) media, the recombinant can be easily selected by complementation, expressed proteins can be specifically engineered for cytoplasmic localization or for extracellular export, and are well suited for large-scale fermentation.

Prokaryotes are the preferred hosts for the initial cloning steps, and are particularly useful for rapid production of large amounts of DNA, for production of single-stranded DNA templates used for site-directed mutagenesis, for screening many mutants simultaneously, and for DNA sequencing of the mutants generated. E. coli strains suitable for the production of the peptides of the present invention include, for example, BL21 carrying an inducible T7 RNA polymerase gene (Studier et al., Methods Enzymol. 185:60-98 (1990)); AD494 (DE3); EB 105; and CB (E. coli B) and their derivatives; K12 strain 214 (ATCC 31,446); W3110 (ATCC 27,325); X1776 (ATCC 31,537); HB101 (ATCC 33,694); JM101 (ATCC 33,876); NM522 (ATCC 47,000); NM538 (ATCC 35,638); NM539 (ATCC 35,639), etc. Many other species and genera of prokaryotes may be used as well. Indeed, the peptides of the present invention can be readily produced in large amounts by utilizing recombinant protein expression in bacteria, where the peptide is fused to a cleavable ligand used for affinity purification.

Suitable promoters, vectors and other components for expression in various host cells are well known in the art and are disclosed, for example, in the textbooks listed above.

Whether a particular cell or cell line is suitable for the production of the polypeptides herein in a functionally active form, can be determined by empirical analysis. For example, an expression construct comprising the coding sequence of the desired molecule may be used to transfect a candidate cell line. The transfected cells are then growth in culture, the medium collected, and assayed for the presence of polypeptide.

Alternatively, the polypeptide sequences may be prepared by chemical synthesis, such as solid phase peptide synthesis. Such methods are well known to those skilled in the art. In general, these methods employ either solid or solution phase synthesis methods, described in basic textbooks, such as, for example, J. M. Stewart and J. D. Young, Solid Phase Peptide Synthesis, 2nd Ed., Pierce Chemical Co., Rockford, Ill. (1984) and G. Barany and R. B. Merrifield, The Peptide: Analysis Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

The GSTM1 and GSTP1 105val polypeptides of the present invention may include amino acid sequence variants of native GST sequences, so long as the polypeptides retain the biological function of the native GSTM1 or GSTP1105val polypeptide. Such amino acid sequence variants can be produced by expressing the underlying DNA sequence in a suitable recombinant host cell, or by in vitro synthesis of the desired polypeptide, as discussed above. The nucleic acid sequence encoding a polypeptide variant is preferably prepared by site-directed mutagenesis of the nucleic acid sequence encoding the corresponding native (e.g. human) polypeptide. Particularly preferred is site-directed mutagenesis using polymerase chain reaction (PCR) amplification (see, for example, U.S. Pat. No. 4,683,195 issued Jul. 28, 1987; and Current Protocols In Molecular Biology, Chapter 15 (Ausubel et al., ed., 1991). Other site-directed mutagenesis techniques are also well known in the art and are described, for example, in the following publications: Current Protocols In Molecular Biology, supra, Chapter 8; Molecular Cloning: A Laboratory Manual., 2nd edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

Compositions and Formulations of the Invention

For therapeutic uses, including prevention, the compounds of the invention can be formulated as pharmaceutical compositions in admixture with pharmaceutically acceptable carriers or diluents. Methods for making pharmaceutical formulations are well known in the art.

Techniques and formulations generally may be found in Remington's Pharmaceutical Sciences, 18th Edition, Mack Publishing Co., Easton, Pa. 1990. See, also, Wang and Hanson “Parenteral Formulations of Proteins and Peptides: Stability and Stabilizers”, Journal of Parenteral Science and Technology, Technical Report No. 10, Supp. 42-2S (1988). A suitable administration format can best be determined by a medical practitioner for each patient individually.

Pharmaceutical compositions of the present invention can comprise the GSTM1 protein or the GSTP1 105val protein, the GSTM1 DNA or GSTP1 105val DNA of the present invention along with conventional carriers and optionally other ingredients.

Suitable forms, in part, depend upon the use or the route of entry, for example oral, transdermal, inhalation, or by injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms that prevent the agent or composition from exerting its effect.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including, but not limited to, oral, intravenous, intraarterial, intraperitoneal, subcutaneous, intranasal or intrapulmonary routes. The desired isotonicity of the compositions can be accomplished using sodium chloride or other pharmaceutically acceptable agents such as dextrose, boric acid, sodium tartrate, propylene glycol, polyols (such as mannitol and sorbitol), or other inorganic or organic solutes.

For systemic administration, injection is preferred, e.g., intramuscular, intravenous, intra-arterial, etc. For injection, the compounds of the invention are formulated in liquid solutions, preferably in physiologically compatible buffers such as Hank's solution or Ringer's solution. Alternatively, the compounds of the invention are formulated in one or more excipients (e.g., propylene glycol) that are generally accepted as safe as defined by USP standards. They can, for example, be suspended in an inert oil, suitably a vegetable oil such as sesame, peanut, olive oil, or other acceptable carrier. Preferably, they are suspended in an aqueous carrier, for example, in an isotonic buffer solution at pH of about 5.6 to 7.4. These compositions can be sterilized by conventional sterilization techniques, or can be sterile filtered. The compositions can contain pharmaceutically acceptable auxiliary substances as required to approximate physiological conditions, such as pH buffering agents. Useful buffers include for example, sodium acetate/acetic acid buffers. A form of repository or “depot” slow release preparation can be used so that therapeutically effective amounts of the preparation are delivered into the bloodstream over many hours or days following transdermal injection or delivery. In addition, the compounds can be formulated in solid form and redissolved or suspended immediately prior to use. Lyophilized forms are also included.

Alternatively, certain molecules identified in accordance with the present invention can be administered orally. For oral administration, the compounds are formulated into conventional oral dosage forms such as capsules, tablets and tonics.

Systemic administration can also be by transmucosal or transdermal. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents can be used to facilitate permeation. Transmucosal administration can be, for example, through nasal sprays or using suppositories.

A preferred route for administration of the compounds of the invention may be inhalation for intranasal and/or intrapulmonary delivery. For administration by inhalation, usually inhalable dry power compositions or aerosol compositions are used, where the size of the particles or droplets is selected to ensure deposition of the active ingredient in the desired part of the respiratory tract, e.g. throat, upper respiratory tract or lungs. Inhalable compositions and devices for their administration are well known in the art. For example, devices for the delivery of aerosol medications for inspiration are known. One such device is a metered dose inhaler that delivers the same dosage of medication to the patient upon each actuation of the device. Metered dose inhalers typically include a canister containing a reservoir of medication and propellant under pressure and a fixed volume metered dose chamber. The canister is inserted into a receptacle in a body or base having a mouthpiece or nosepiece for delivering medication to the patient. The patient uses the device by manually pressing the canister into the body to close a filling valve and capture a metered dose of medication inside the chamber and to open a release valve which releases the captured, fixed volume of medication in the dose chamber to the atmosphere as an aerosol mist. Simultaneously, the patient inhales through the mouthpiece to entrain the mist into the airway. The patient then releases the canister so that the release valve closes and the filling valve opens to refill the dose chamber for the next administration of medication. See, for example, U.S. Pat. No. 4,896,832 and a product available from 3M Healthcare known as Aerosol Sheathed Actuator and Cap.

Another device is the breath actuated metered dose inhaler that operates to provide automatically a metered dose in response to the patient's inspiratory effort. One style of breath actuated device releases a dose when the inspiratory effort moves a mechanical lever to trigger the release valve. Another style releases the dose when the detected flow rises above a preset threshold, as detected by a hot wire anemometer. See, for example, U.S. Pat. Nos. 3,187,748; 3,565,070; 3,814,297; 3,826,413; 4,592,348; 4,648,393; 4,803,978.

Devices also exist to deliver dry powdered drugs to the patient's airways (see, e.g. U.S. Pat. No. 4,527,769) and to deliver an aerosol by heating a solid aerosol precursor material (see, e.g. U.S. Pat. No. 4,922,901). These devices typically operate to deliver the drug during the early stages of the patient's inspiration by relying on the patient's inspiratory flow to draw the drug out of the reservoir into the airway or to actuate a heating element to vaporize the solid aerosol precursor.

Devices for controlling particle size of an aerosol are also known, see, for example, U.S. Pat. Nos. 4,790,305; 4,926,852; 4,677,975; and 3,658,059.

For topical administration, the compounds of the invention are formulated into ointments, salves, gels, or creams, as is generally known in the art.

If desired, solutions of the above compositions can be thickened with a thickening agent such as methyl cellulose. They can be prepared in emulsified form, either water in oil or oil in water. Any of a wide variety of pharmaceutically acceptable emulsifying agents can be employed including, for example, acacia powder, a non-ionic surfactant (such as a Tween), or an ionic surfactant (such as alkali polyether alcohol sulfates or sulfonates, e.g., a Triton).

Compositions useful in the invention are prepared by mixing the ingredients following generally accepted procedures. For example, the selected components can be mixed simply in a blender or other standard device to produce a concentrated mixture which can then be adjusted to the final concentration and viscosity by the addition of water or thickening agent and possibly a buffer to control pH or an additional solute to control tonicity.

The amounts of various compounds for use in the methods of the invention to be administered can be determined by standard procedures. Generally, a therapeutically effective amount is between about 100 mg/kg and 10⁻¹² mg/kg depending on the age and size of the patient, and the disease or disorder associated with the patient. Generally, it is an amount between about 0.05 and 50 mg/kg, more preferably between about 1.0 and 10 mg/kg for the individual to be treated. The determination of the actual dose is well within the skill of an ordinary physician.

The compounds of the present invention may be administered in combination with one or more further therapeutic agent for the treatment of IgE-mediated allergic diseases or conditions.

Such further therapeutic agents include, without limitation, corticosteroids, .beta.-antagonists, theophylline, leukotriene inhibitors, allergen vaccination, soluble recombinant human soluble IL-4 receptors (Immunogen), anti-IL-4 monoclonal antibodies (Protein Design Labs), and anti-IgE antibodies, such as the recombinant human anti-IgE monoclonal antibody rhuMAb-E25 (Genentech, Inc.)(see, e.g. Barnes, The New England Journal of Medicine 341:2006-2008 (1999)). Thus the compounds of the present invention can be used to supplement traditional allergy therapy, such as corticosteroid therapy performed with inhaled or oral corticosteroids.

Gene Therapy

Nucleic acid encoding the GSTM1 or GSTP1 105val proteins may also be used in gene therapy. In gene therapy applications, genes are introduced into cells in order to achieve in vivo synthesis of a therapeutically effective genetic product, for example for replacement of the less active or inactive gene. “Gene therapy” includes both conventional gene therapy where a lasting effect is achieved by a single treatment, and the administration of gene therapeutic agents, which involves the one time or repeated administration of a therapeutically effective dNA or mRNA. It has already been shown that short oligonucleotides can be imported into cells where they act, despite their low intracellular concentrations caused by their restrictive uptake by the cell membrane. The oligonucleotides can be modified to enhance their uptake, e.g. by substituting their negatively charge phosphodiester groups by uncharged groups.

There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liosomes, electroporation, microinjection, cell fusion DEAE-dextran, the calcium phosphate precipitation method etc. The currently preferred in vivo gene transfer techniques include transfection with viral(typically retroviral) vectors and viral coat protein-liposome mediated transfection (Dzau et al., trends in Biotechnology 11, 205-210 (1993). In some situations it is desirable to provide the nucleic acid source with an agent that targets the target cells, such as an antibody specific for a cell surface membrane protein or the target cell, a ligand for a receptor on the target cell, etc. Where liposomes are employed proteins which bind to a cells surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described for example, by Wu et al., J. Bio. Chem. 262: 4429-4432 (1987) and Wagner et al., Proc. Natl. Acad. Sci USA 87 3410-3414 (1990). for review of gene marking and gene therapy protocols see Anderson et al., Science 256: 808-813 (1992).

### II. EXAMPLES

### Example 1

### Diesel Exhaust Particles

Participants

We recruited 19 non-smoking volunteers (seven males and 12 females) in Los Angeles, Calif., USA. All had a positive epicutaneous skin test (>4 mm wheal with surrounding erythema) to short ragweed (an allergen not present in the Los Angeles region) and an allergy history consistent with allergic rhinitis. Based on interview responses, none of the volunteers had any atypical exposure to pollutants and no air pollution alerts in the Los Angeles area were reported during the study periods. None of the participants reported a respiratory infection in the previous 4 weeks and none had used topical or systemic steroids in the 3 months before the study or oral antihistamines for the previous week. None had ever received allergy immunotherapy. All participants were asked to fill out symptom score cards 2 days before and throughout the study period. All studies were approved by the human subject protection committee of the University of California at Los Angeles and all participants gave written informed consent.

TABLE 1
Participants' characteristics

| | Participants (N = 19) |
|---|---|
| Sex | |
| Women | 12 |
| Men | 7 |
| Age (years) | |
| 20-25 | 7 |
| 26-30 | 8 |
| 30-34 | 4 |
| Ethnic origin | |
| White | 8 |
| Hispanic | 5 |
| African American | 1 |
| Asian | 5 |
| Genotype | |
| GSTM1 | |
| Null | 14 |
| Present | 5 |
| GSTT1 | |
| Null | 9 |
| Present | 10 |
| GSTP1* | |
| I/I | 13 |
| I/V | 6 |
| V/V | 0 |

*A105G polymorphism codes replacement of I by V.

Procedures

A single blind, randomised, placebo-controlled crossover study was conducted. (Gilliland et al., Lancet, Jan. 10, 2004) Nasal washes and provocation challenges were done as described previously (Diaz-Sanchez J. Immunol 1997 158:2406-13; Diaz-Sanchez J. Allergy Clin Immunol 1996 98:114-23; Naclerio et al., J. Allergy Clin Immunol 1997 100:505-10) To establish a positive allergen challenge level, we gave participants increasing intranasal doses of short ragweed (Amb al, Hollister Stier/Baxter, Irwindale, Calif.) starting at 10 allergic units and increasing in ten-fold steps until a symptom score of 5 (of a possible 12 points) was achieved. This dose of allergen was used in the subsequent challenges. Participants then underwent two subsequent challenges that were at least 6 weeks apart. At those times, they were challenged intranasally in a randomized, blinded, cross-over fashion with either allergen plus placebo (300 μL saline), or allergen plus 0-3 mg diesel exhaust particles (in 300 μL saline). The diesel exhaust particles used had been generated in 2001 from a light-duty four-cylinder diesel engine (4JBI type, Isuzu Automobile Company, Japan) using standard diesel fuel. Diesel exhaust particles stocks are stored under nitrogen in the dark and working volumes are stored at −80° C. in the dark. This storage prevents oxidation or loss of volatile chemicals.

Nasal washes (5 mL normal saline in each nostril) were done immediately before, and at 10 min, 24 h, and 72 h after the challenge, as described previously. (Diaz-Sanchez J. Immunol 1997 158:2406-13; Diaz-Sanchez J. Allergy Clin Immunol 1996 98 114-23; Naclerio et al., J. Allergy Clin Immunol 1997 100:505-10). Results are reported for the 10 min post-challenge time point for histamine and the 24-h post-challenge timepoint for other responses since the respective responses have been previously shown to be maximum at these time points.

We collected nasal washes, centrifuged them at 350 g for 10 min at 4° C., and separated the aqueous supernatants from the cell pellets. Total and ragweed specific IgE in nasal washes were measured by isotype specific ELISAs as described previously (Diaz-Sanchez J. Immunol 1997 158:2406-13; Diaz-Sanchez et al., J Clin Invest 1994 94:1417-25). We measured histamine concentrations with a commercial assay (Immunotech, Brea, Calif.) following the manufacturers' instructions. The sensitivity of the assay was 0.5 nmol/L. The cytokines interleukin 4 and interferon γ were measured with commercial ELISA kits (BD Pharmingen, San Diego, Calif.) following the manufacturers' instructions. For the purposes of statistical analyses, the lower limit of detection for each assay was used for patients with values below the limit of detection.

We obtained buccal cells from participants as a source of genomic DNA. Buccal cells were collected from participants as a source of germline DNA for genotyping assays. Participants were provided with a new toothbrush and instructed to brush their teeth. A second toothbrush was provided with instructions to gently brush the buccal mucosa. The brush was then placed in a leak proof container that was filled with an alcohol-based fixative. Participants then swished liquid throughout their mouths and expelled the fluid into a container.

Buccal cell suspensions were centrifuged at 2000 g on the day they were received in the laboratory. The pellets were stored frozen at −20° C. until used for DNA extraction, at which time they were resuspended and incubated in 600 μl of lysis solution from a PUREGENE DNA isolation kit (cat #D-5000, GENTRA, Minneapolis, Minn.) containing 100 μg/ml proteinase K overnight at 55° C. DNA extraction was performed according to manufacturer's recommendations. The DNA samples were resuspended in aqueous solution and stored at −20° C.

Genotypes for GSTM1 were determined using real-time PCR. Polymorphic GSTM1 gene sequences were analyzed using a TaqMan 7700 (Applied Biosystems, city). The forward and reverse primers for GSTM1 were 5′-CTTGGAGGAACTCCCTGAAAAG-3′ (SEQ. ID NO:1) and 5′-TGGAACCTCCATAACACGTGA-3′ (SEQ ID NO:2) respectively. The probe used for GSTM1 was a 6-carboxyfluorescein (FAM) labeled and 5-carboxytetramethylrhodamine (TAMRA) labeled probe (6FAM-AAGCGGCCATGGTTTGCAGG-TAMRA. (SEQ ID NO:3)). The denaturation step during the first cycle was 10-minutes at 95° C. For the remainder of the amplification reaction, a 2-cycle protocol characterized by a 15-second incubation at 95° C. followed by a one-minute incubation at 60° C. was used.

GSTT1 genotypes were determined using real-time PCR using a TaqMan 7700 (Applied Biosystems, Foster City, Calif.). The forward and reverse primers for GSTT1 were 5′-GTGCAAACACCTCCTGGAGAT-3′ (SEQ ID NO:4) and 5′AGTCCTTGGCCTTCAGAATGA-3′ (SEQ ID NO:5) respectively. The probe used for GSTT1 was a 6-carboxyfluorescein (FAM) labeled and 5-carboxy-tetramethyl-rhodamine (TAMRA) labeled probe (6FAM-ATGCTGCCCATCCCTGCCC-TAMRA (SEQ ID NO:6)). The presence or absence of a fluorescent amplification signal was used as an indication of whether the GSTT1 alleles were present or absent in a particular genomic DNA sample. The denaturation step during the first cycle was 10 minutes at 95° C. For the remainder of the amplification reaction, we used a 2-cycle protocol characterized by a 15-second incubation at 95° C. followed by a 1-minute incubation at 60° C.

The presence or absence of a fluorescent amplification signal was used as an indication of whether the GSTM1 and GSTT1 alleles were present or absent in a genomic DNA sample. Samples showing no signal or late cycle number for start of amplification for either one of these alleles were repeated and further analyzed with primers and probes for the actin gene to verify the presence of amplifiable DNA.

Analysis of the single nucleotide polymorphism at codon 105 in the GSTP1 gene was performed using allele-specific probes. The forward and reverse primers for GSTP1 were 5′-CCTGGTGGACATGGTGAATG-3′ (SEQ ID NO:7) and 5′TGCTCACACCATAGTTGGTGTAGATGA-3′ (SEQ ID NO:8) respectively. The allele-specific probes were 6-carboxyfluorescein (FAM) labeled and minor groove-binding fluorescent quencher (MGBNFQ) labeled (Applied Biosystems Inc.). The probes were (6FAM-TGCAAATACATCTCCCT-MGBNFQ (allele 1) (SEQ ID NO:9)and VIC-CTGCAAATACGTCTCC-MGBNFQ (allele 2) (SEQ ID NO:10). The fact that the wavelength of the fluorescent label was different in the 2 probes allowed distinguishing between amplification products from each allele in a single reaction. The conditions for the 2-cycle PCR were similar to those used for GSTM1 polymorphisms. Samples showing no signal or late cycle number for start of amplification for either one of these alleles were repeated and further analyzed with primers and probes for the actin gene to verify the presence of amplifiable DNA.

Statistical Analysis

First, we assessed the distributions of allergen-specific IgE, interleukin 4, interferon γ, the ratio of interleukin 4 to interferon-y, and histamine, and found that each was skewed and did not follow a normal distribution. Therefore, we compared median concentrations and median differences of allergen-specific IgE, interleukin 4, interferon-γ, and histamine after allergen challenge and after allergen plus diesel exhaust particles challenge. We also used the median values to assess the ratio of interleukin 4, to interferon γ. We tested the hypothesis with non-parametric Wilcoxon signed-rank tests for difference in the median values. We also provide means and t tests for differences in mean concentrations for completeness. The effect of GSTM1, GSTT1, and GSTP1 genotypes on allergen-specific IgE, histamine, interleukin 4, and interferon γ concentrations after allergen alone or diesel exhaust particles plus allergen were assessed by comparisons of median responses between different genotypes and statistical testing was again done with Wilcoxon signed-rank tests for median differences. All analyses were done with SAS software version 8.0 and all reported p values are based on a two-sided alternative hypothesis. p values were judged significant if they were less than 0.05.

Results

Table 1 shows the participants' characteristics. GSTM1 and GSTT1 null genotypes were present in 74% (14 of 19) and 47% (nine of 19) of patients, respectively. Most (68%, 13 of 19) patients were homozygous for the GSTPI I105 wild-type allele and none was homozygous for the GSTP1 V105 variant allele. We selected the patients on the basis of their nasal allergy status, which probably explains the genotype distribution that differs from that seen in general population studies.

We have previously reported that diesel exhaust particles enhance allergen-driven, IgE, histamine, and interleukin 4 responses while decreasing production of interferon γ (Mcconnell et al., Environ Health Perspect 1999: 107 757-60; Diaz-Sanchez et al., J. Allergy Clin Immunol 2001 106:1140-46) Diesel exhaust particles greatly increased the allergic response after nasal challenge. On exposure to diesel exhaust particles plus allergen, nasal allergen specific IgE concentrations increased more than ten-fold compared with allergen alone (Table 2) for all participants. Histamine concentrations were also about five-fold higher after diesel exhaust particles plus allergen than after allergen alone (Table 2). Compared with allergen challenge alone, exposure to diesel exhaust particles plus allergen increased interleukin 4 and decreased interferon γ concentrations consistent with an enhancement of the allergic response (Table 2).

TABLE 2
Nasal responses after exposure to allergen plus clean
air or allergen plus diesel exhaust particles

| | Clean air | DEP and | | |
|---|---|---|---|---|
| | and allergen | allergen | Difference | p* |
| lgE (U/mL) | 9.8 (6.4) | 121.2 (134.1) | 111.4 (129.7) | 0.002 |
| Interleukin | 0.3 (0.1) | 6.0 (5.0) | 5.7 (4.9) | <0.0001 |
| 4 (U/mL) | | | | |
| Interferon | 1.2 (0.6) | 0.6 (0.5) | −0.6 (0.8) | 0.002 |
| γ (ngVL) | | | | |
| Interfer- | 4.8 (2.7)† | 0.6 (1.4)† | 0.1 (0.3)† | <0.0001 |
| on -γ/ | | | | |
| Interleukin 4 | | | | |
| Histamine | 3.1 (1.3) | 15.0 (7.4) | 11.8 (7.0) | <0.0001 |
| (nmol/L) | | | | |

DEP = diesel exhaust particles.
Values are mean (SD).
*Paired t tests for means.
†Value is mean (SD) ratio

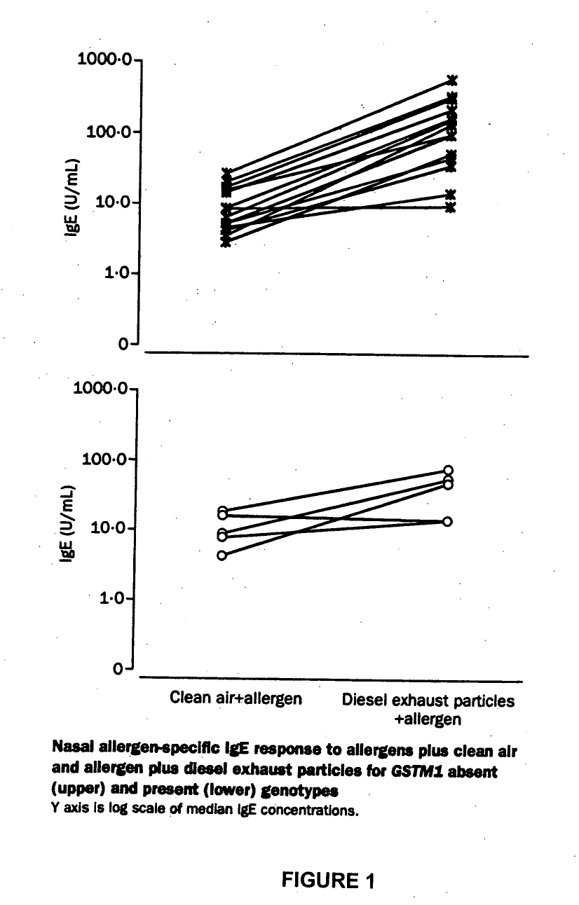

Table 3 and FIG. 1 show that individuals with either a null GSTM1 or homozygous GSTP1 I105 genotypes had much higher nasal IgE responses to diesel exhaust particles than to the allergen alone. Compared with participants with GSTM1 present genotype, those with GSTM1 null had a significantly larger increase in antiragweed IgE (median 102.5 U/mL [1.0-510.5] vs 45.5 U/mL [−1.5-60.6], p=0.03) after diesel exhaust particles plus allergen challenge. Compared with participants with a GSTP1 V105 variant, those with the homozygous wild-type I105 GSTP1 genotype had a significantly larger increase in allergic specific IgE after diesel exhaust particles plus allergen challenge (median 120.3 U/mL [6.7-510.5] vs 27.7 U/mL [1.5-60.6], p=0.03). By contrast, GSTT1 genotype was not associated with diesel exhaust particles-enhanced IgE responses. None of the GSTs modified the allergic response to allergen challenge alone.

TABLE 3
Effects of GSTM1, GSTT1 and GSTP1 genotype on nasal IgE (U/mL) and histamine (nmol/l when
exposed to allergen plus clean air or allergen plus diesel exhaust particles (DEP)

| | GSTM1 | | | GSTT1 | | | GSTP1 | | |
|---|---|---|---|---|---|---|---|---|---|
| | Null | Present | | | Present | | | I/V | |
| | (n = 14) | (n = 5) | P | Null (n = 9) | (n = 10) | P | I/I (n = 13) | (n = 6) | P |
| IgE | | | | | | | | | |
| Clean air | 6.9 | 8.9 | 0.40 | 7.9 | 7.8 | 0.57 | 7.8 | 8.4 | 1.00 |
| and | (2.6-24.3) | (4.3-18.8) | | (3.8-24.3) | (2.6-18.7) | | (3.2-24.3) | (2.6-18.8) | |
| allergen | | | | | | | | | |
| DEP and | 106.6 | 49.8 | 0.15 | 89.5 | 49.3 | 0.35 | 123.5 | 31.5 | 0.02 |
| allergen | (8.8-534.8) | (14.2-79.4) | | (13.3-534.5) | (8.8-312.5) | | (14.5-534.8) | (8.8-79.4) | |
| Difference | 102.5 | 45.5 | 0.03 | 84.7 | 45.9 | 0.35 | 120.3 | 27.7 | 0.03 |
| | (1.0-510.5) | (−1.5-60.6) | | (9.1-510.5) | (−1.5-293.8) | | (6.7-510.5) | (−1.5-60.6) | |
| Histamine | | | | | | | | | |
| Clean air | 2.9 | 2.8 | 0.96 | 2.8 | 2.9 | 0.65 | 2.9 | 3.0 | 0.63 |
| and | (1.3-5.9) | (1.9-6.7) | | (2.2-4.3) | (1.3-6.7) | | (1.3-6.7) | (1.9-6.0) | |
| allergen | | | | | | | | | |
| DEP and | 16.9 | 9.8 | 0.08 | 15.7 | 16.4 | 1.00 | 17.2 | 8.5 | 0.04 |
| allergen | (2.9-27.6) | (3.1-19.0) | | (7.3-25.8) | (2.9-27.6) | | (6.2-27.6) | (2.9-25.5) | |
| Difference | 14.0 | 7.4 | 0.02 | 12.9 | 12.7 | 0.97 | 13.8 | 5.2 | 0.01 |
| | (−0.2-24.7) | (1.2-12.3) | | (3.0-21.8) | (−0.2-24.7) | | (3.1-24.7) | (−0.2-19.6) | |

Values are median (range). p values calculated with Wilcoxon rank sums test.

Parallel effects of the GSTM1 null and I105 GSTP1 genotypes were seen with histamine release enhanced by diesel exhaust particles (Table 3). In participants with a null GSTM1, histamine concentrations were significantly higher after diesel exhaust particles plus allergen challenge than in those with the functional GSTM1 genotype (Table 3). Similarly, those with the GSTP1 I105 variant had higher histamine concentrations after diesel exhaust particles plus allergen challenge than did those with the homozygous genotype (Table 3).

TABLE 4
IgE (U/mL) and histamine (nmol/L) differences
by joint GSTM1 and GSTP1 genotype

| | | | | Histamine |
|---|---|---|---|---|
| GSTM1 | GSTP1 | n | IgE difference | difference |
| Present | I/I | 2 | 26.1 (6.7-45.5) | 7.73 (3.13-12.32) |
| Present | I/V | 3 | 48.9 (−1.5-60.6) | 7.44 (1.22-7.48) |
| Null | I/I | 11* | 137 0 (29.9-510.5) | 14.33 (8.14-24.67) |
| Null | I/V | 3 | 9.1 (1.0-46.2) | 2.98 (−0.22-19.59) |

Values are median (range). *p = 0.0034 for IgE and p = 0.0073 for histamine calculated by the Wilcoxon test comparing GSTM1 null/GSTP1 1/1 with the other three genotype groups combined.

The joint GSTM1 and GSTP1 genotype seems to be an important determinant of response to diesel exhaust particles (Table 4). Of the 14 participants who were allergic and had the GSTM1 null genotype, 11 had the normal GSTP1 I/I genotype. Those with this joint genotype had significantly higher allergic responses to diesel exhaust particles than the other genotypes combined. Our sample size does not allow a full assessment of gene-gene-environment interaction for the GSTs in this study.

Interleukin 4 responses to diesel exhaust particles did not significantly differ between the GSTM1 null and GSTP1 I/I genotype and the GSTM1 present and the GSTP1 I/V genotype, respectively (Table 5). The concentrations of interferon-γ and the ratio of interleukin 4 to interferon-γ did not show consistent patterns by genotype.

TABLE 5
Effects of GSTM1, GSTT1, and GSTP1 genotype comparing differences and ratio
of responses to allergen plus diesel exhaust with allergen plus clean air

| | GSTM1 | | | GSTT1 | | | GSTP1 | | |
|---|---|---|---|---|---|---|---|---|---|
| | Null | Present | | Null | Present | | I/I | V/V | |
| | (n = 14) | (n = 5) | P | (n = 9) | (n = 10) | P | (n = 13) | (n = 6) | P |
| Interferon γ | −0.5 | −0.8 | 0.75 | −0.9 | −0.2 | 0.18 | −0.8 | −0.6 | 1.00 |
| (nVL) | (−1.9 to 0.2) | (−1.8 to 0.5) | | (−1.9 to 0.2) | (−1.8 to 0.5) | | (−1.9 to 0.5) | (−1.7 to 0.3) | |
| Interleukin 4 | 5.6 | 2.8 | 0.15 | 5.1 | 4.7 | 0.97 | 5.1 | 3.3 | 0.51 |
| (U/mL) | (0.0 to 15.0) | (0.0 to 8.1) | | (0.0 to 9.2) | (0.0 to 15.0) | | (0.0 to 15.0) | (0.7 to 14.4) | |
| Interferon | 0.02 | 0.03 | 0.49 | 0.02 | 0.05 | 0.65 | 0.02 | 0.03 | 0.69 |
| γ/interleukin 4 | (0.0 to 0.44) | (0.0 to 1.32) | | (0.0 to 0.44) | (0.0 to 1.32) | | (0.0 to 1.32) | (0.0 to 0.28) | |

Values are median (range).

### Example 2

### Environmental Tobacco Smoke

Subjects

A total of 19, non-smoking volunteers (7 males and 12 females) age 20 to 34 years old were recruited in Los Angeles, Calif. (Table 6). All had been shown to have an allergy history consistent with allergic rhinitis and a positive intradermal skin test to short ragweed. While all subjects showed positive skin tests for other allergens, all were asymptomatic and none complained of symptoms during the course of the study. In addition, all subjects who were challenged intranasally with the ragweed allergen Amb a I displayed immediate allergic symptoms such as sneezing, runny nose, and ocular itching. The subjects did not take any medication for the 3 days prior to or during the duration of the study. None of the volunteers cohabited with smokers or had any known extensive or extraordinary exposure to pollutants. Short ragweed was used as the antigen in the nasal challenges since it is not present in the Los Angeles area and the cross-reacting western ragweed is a minor allergen. Ragweed IgE levels in nasal lavages were very low or undetectable prior to challenge. The research was approved by the Human Subject Protection Committees of the University of California at Los Angeles and Los Amigos Research and Education Institute, Rancho Los Amigos National Rehabilitation Center, Los Angeles. All subjects provided written consent.

TABLE 6
Selected characteristics for 19 study participants

| | n | % |
|---|---|---|
| Total | 19 | 100.0% |
| Gender | | |
| Female | 12 | 63.2% |
| Male | 7 | 36.8% |
| Age (years) | | |
| 20-25 | 7 | 36.8% |
| 26-30 | 8 | 42.1% |
| 30-34 | 4 | 21.1% |
| Ethnicity | | |
| Caucasians | 8 | 42.1% |
| Hispanics | 5 | 26.3% |
| African Americans | 1 | 5.3% |
| Asians | 5 | 26.3% |
| I. Genotype | | |
| 1. GSTM1 | | |
| Null | 14 | 73.7% |
| Present | 5 | 26.3% |
| GSTP1 Ile105Val (A→G) | | |
| Ile/Ile | 13 | 68.4% |
| Ile/Val | 6 | 31.6% |
| Val/Val | 0 | 0.0% |

Generation of Environmental Tobacco Smoke (ETS) and Controlled Exposure

The exposure chamber (700 cubic feet) maintained controlled temperature (70° F.), humidity (50%) and ventilation (8 exchanges/hour) during the exposures, which lasted for two hours. During this time subjects rested and refrained from vigorous activity. ETS was generated from the side-stream smoke of 1R4F cigarettes (University of Kentucky Tobacco and Health Research Institute; Lexington, Ky.). ETS consists primarily (95%) of side-stream smoke (emitted from the burning zone) and also (4%) smolder stream smoke (emitted from the puffing zone). (Johnson et al., Crit. Rev. Toxicol 1990; 20:369-95). Each of these filtered reference cigarettes contains 9.2 mg tar and 0.8 mg nicotine. (Institute TaHR. The Reference Cigarette. Lexington, Ky.: The University of Kentucky Printing Services; 1990) In order to maintain proper moisture levels, the cigarettes were stored in a sealed plastic bag at 4° C. They were immediately brought to room temperature 15 minutes before needed and lit in a RM G1 Borgwaldt smoking machine (Hamburg, Germany). This automated smoking machine was set to conform to the Federal Trade Commission guidelines for the generation of side-stream smoke: one inhalation every 55 seconds with an inhalation/exhaust cycle of 5 sec duration. Mainstream smoke was captured and excluded from entering into the system. A total of five cigarettes were “smoked” by the machine in each two-hour period.

The levels of carbon monoxide in the chamber were continuously monitored and were never higher than 5 ppm during the exposure. The mean PM level was 310 μg/m³. The ETS exposure was well tolerated by all subjects. Five of the subjects reported mild irritation of the throat and eyes during exposure but these symptoms did not persist for more than 5 minutes. No other adverse symptoms were noted. Clean air exposures were performed in the same manner with the absence of smoke generation. The mean PM level during clean air exposure was 46 μg/m³. All exposures were done at the same time of day (between 9 and 11 am) to avoid diurnal variation.

Allergen Challenge and Nasal Lavage

Allergen challenge was performed as previously detailed (Diaz-Sanchez et al., J Immunol. 1997; 158:2406-13; Diaz-Sanchez D et al., J Allergy Clin Immunol 2001; 106:1140-6). Briefly: At least 30 days before each clean air/ETS exposure, a dose of allergen that would elicit allergic symptoms was established for each subject. This was done by spraying the nose of allergic subjects with increasing doses of an extract of ragweed containing a known amount of the antigen Amb a 1 (Hollister Stier/Baxter, Irwindale, Calif.). The starting dose was 10 AU and this was increased until a symptom score of five (out of 12) was achieved in our allergic symptom severity score system. (Diaz-Sanchez et al., J Immunol. 1997; 158:2406-13; Diaz-Sanchez D et al., J Allergy Clin Immunol 2001; 106:1140-6). Subjects then returned for two subsequent visits that were spaced at least six weeks apart. In these visits allergen challenge was performed following either clean air or ETS exposure. Subjects were challenged with the established allergen dose immediately after the nasal lavage performed following clean air/ETS exposure.

Nasal lavage is a well established procedure which has been used for over 20 years to study the effect of pollutants on the upper airway (Koren H S. Toxicology 1990; 60:15-25. and were performed as previously described (Diaz-Sanchez D, et al., J Clin Invest. 1994; 94:1417-25; Diaz-Sanchez D, et al., J Allergy Clin Immunol. 1996; 98:114-23). Briefly, 5 mL of normal saline was delivered into each nostril of the subjects and after 10 seconds, the wash fluid was collected. The subjects then performed four subsequent nasal washes. The tubes were centrifuged at 350 g for 10 minutes at 4° C. and the aqueous supernatants separated from the cell pellets and stored at −20° C. until needed.

Immunoassays

The levels of IgE, IgG, IgG4, and IgA in the supernatants were measured by isotype specific ELISAs as previously described with minor modifications. (Diaz-Sanchez D, et al., J Immunol. 1997; 158:2406-13; Diaz-Sanchez D, et al., J Clin Invest. 1994; 94:1417-25;: Macy et al., FASEB J 1988; 2:300-10). All samples were run in duplicate and repeated if there was more than a 10% variation between the duplicates. Ragweed-specific IgE, IgG and IgG4 were determined as previously reported using the same procedure as for total Ig isotypes except that an amplification system previously described was used with minor modifications. (Diaz-Sanchez D, et al., J Immunol. 1997; 158:2406-13). Cytokines were measured using commercial ELISA kits (BD Pharmingen, San Diego, Calif.) as per manufacturers' instructions.

Histamine was measured in nasal lavages performed prior and immediately after ETS/clean air exposure and 10 minutes after allergen exposure. Histamine levels in nasal washes were measured using a commercial assay (Immunotech, Brea, Calif.) as per manufacturer's instructions. The sensitivity of the assay was 0.5 nM.

Buccal cells were obtained as described in Example 1 from the participants. Genotypes for GSTM1 were obtained as described in Example 1.

Results

Allergen-specific IgE is the hallmark of allergic disease. ETS promoted the production of ragweed-specific IgE. As expected challenge with ragweed following clean air (control) exposure resulted in a significant increase in ragweed-specific IgE at days 1, 4 and 8. However, the levels detected in nasal lavage fluids after ragweed challenge following ETS exposure dwarfed these responses. Four days after exposure to ragweed plus ETS levels were up to 65 fold higher in some subjects than following clean air/ragweed challenge (mean=199 U/mL vs. 12 U/mL). This resulted in highly significant differences between the two challenge protocols (p<0.005 and p<0.001 for Day 1 and Day 4 respectively, paired t-test).

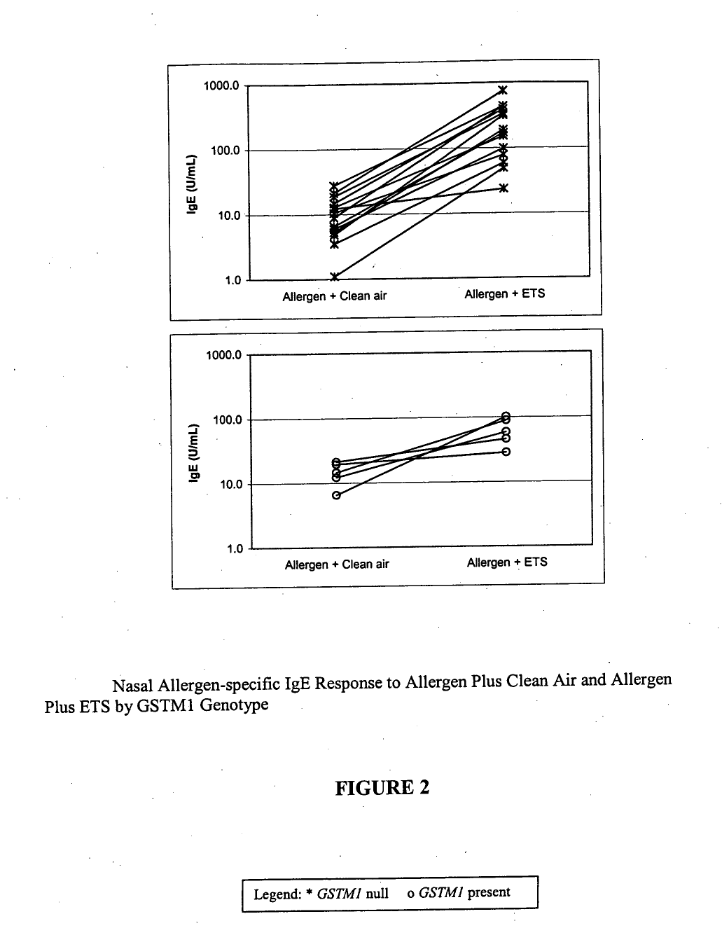

FIG. 2 shows that individuals with a GSTM1 null genotype had a much higher nasal IgE response to ETS plus allergen than to allergen alone compared to participants with GSTM1 present genotype.

Levels of ragweed specific-IgG4 were significantly higher following ETS/ragweed exposure than clean air/ragweed exposure (e.g. after 4 days mean levels were 26.7 U/mL vs. 5.6 U/mL). In contrast, no differences were observed between the two exposure regimes in levels of other antibody types (data not shown). Thus for both ragweed-specific IgG and IgA, levels measured in nasal washes were the same after challenge with ETS/ragweed or with clean air/ragweed (p>0.05, paired t-test). As expected exposure to ETS in the absence of allergen did not result in the formation of any allergen-specific antibodies (data not shown).

ETS synergised with allergen to produce a local Th2 cytokine milieu, a response characteristic of an enhanced allergic response and critical to allergic inflammation. Following challenge of allergic subjects with allergen alone we observed little or no change in nasal cytokine levels. In contrast, if allergen-challenge was performed following ETS exposure, there was a rise in IL-4, IL-5 and IL-13 levels in nasal washes obtained 24 hours later. The levels of these cytokines were significantly greater at this time than following challenge with allergen alone (e.g. IL-5 mean=4.8 pg/mL vs. 0.3 pg/mL, p<0.01). In contrast, IFN-γ levels were not significantly changed by ETS plus allergen exposure. ETS exposure alone did not significantly enhance IL-4, IL-5, IL-13 or IFN-γ, in lavages obtained 24 hours later.

Histamine levels were measured in nasal lavage fluid obtained 10 min following challenge with ragweed allergen. Significantly higher levels were measured when subjects were pre-exposed to ETS than clean air. Baseline levels of histamine were virtually identical in all challenge days. Following clean air/ragweed challenge there was a 7.7 fold rise in mean histamine from baseline values (4.02 vs. 0.52 nM). In contrast, this was significantly less than the 25.1 fold increase observed following ETS/ragweed challenge. In the absence of allergen provocation no changes in histamine levels were observed. Thus 2 h. ETS or clean air exposure alone did not result in elevation of histamine above baseline levels.

The patents and publications listed herein describe the general skill in the art and are hereby incorporated by reference in their entireties for all purposes and to the same extent as if each was specifically and individually indicated to be incorporated by reference. In the case of any conflict between a cited reference and this specification, the specification shall control.

While the present application has been described in the context of embodiments illustrated and described herein, the invention may be embodied in other specific ways or in other specific forms without departing from its spirit or essential characteristics. Therefore, the described embodiments are to be considered in all respects as illustrative and not restrictive. The scope of the invention is therefore indicated by the appended claims rather than the foregoing description, and all changes that come within the meaning and range of equivalency of the claims are to be embraced within their scope.

1. A method for determining whether a subject is susceptible to enhancement of allergen-induced hypersensitivity reaction by airborne pollutants comprising genotyping said subject for genes selected from the group consisting of GSTM1 or GSTP1 genes.

2. The method of claim 1 wherein said reaction is a type I allergic hypersensitivity reaction

3. The method of claim 1 wherein said reaction is an increase in production of IgE to allergens.

4. The method of claim 1 wherein the airborne pollutants are selected from the group consisting of traffic pollutants and indoor pollutants.

5. The method of claim 4 wherein the airborne pollutants are diesel exhaust particles.

6. The method of claim 4 wherein the airborne pollutants are tobacco smoke particles.

7. The method of claim 1 wherein the gene is GSTM1.

8. The method of claim 7 wherein the genotype is GSTM1 null genotype.

9. The method of claim 1 wherein the gene is GSTP1.

10. The method of claim 9 wherein the genotype is GSTP1 105 Ile/Ile genotype.

11. The method of claim 1 wherein the said step of assaying is selected from the group consisting of probe hybridization, restriction fragment length polymorphism (RFLP) analysis, minisequencing, MALS-TOF, SINE, heteroduplex analysis, single strand conformational polymorphism (SSCP), denaturing gradient gel electrophoresis (DGGE) and temperature gradient gel electrophoresis (TGGE).

12. The method of claim 1 further comprising the step of amplifying the amount of the GSTP1 or GSTM1 gene or a portion thereof which contains said polymorphism.

13. The method of claim 12 wherein said amplification includes the step of :selecting a forward and a reverse sequence primer capable of amplifying a region of the GSTP1 or GSTM1 gene which contains a polymorphic site.

14. The method of claim 13 wherein the forward and reverse primers for the GSTP1 gene are selected from the group comprising the sequences:

| | |
|---|---|
| 5′-CCTGGTGGACATGGTGAATG-3′ | (SEQ ID NO:7) |
| and | |
| 5′TGCTCACACCATAGTTGGTGTAGATGA-3′: | (SEQ ID NO 8) |

15. The method of claim 13 wherein the forward and reverse primers for the GSTM1 gene are selected from the group comprising the sequences:

| | |
|---|---|
| 5′-CTTGGAGGAACTCCCTGAAAAG-3′ | (SEQ ID NO:1) |
| and | |
| 5′-TGGAACCTCCATAACACGTGA-3′. | (SEQ ID NO:2) |

16. A kit for determining whether a subject is susceptible to enhancement of allergen-induced hypersensitivity reaction comprising a set of primers to amplify a gene selected from the group consisting of the GSTM1 gene or the GSTP1 gene, DNA polymerase and a labeled probe to identify the GSTM1 polymorphism or the GSTP1 polymorphism.

17. A pharmaceutical composition comprising a protein or polypeptide selected from the group consisting of GSTM1 protein, GSTP1 105val protein GSTM1 nucleic acid coding sequence and GSTP1 nucleic acid coding sequence in admixture with a pharmaceutically acceptable excipient or ingredient.

18. A method for the treatment and prevention of enhancement of allergen induced hypersensitivity reaction by airborne pollutants in a subject where the subject is administered a polypeptide selected from the group consisting of GSTM1 protein or GSTP1 105val protein.

19. The method of treatment of claim 18 comprising administering at least one, or alternatively multiple times, an effective amount of at least one polypeptide or protein selected from the group consisting of GSTM1 protein or GSTP1 105val protein to a subject diagnosed with or at risk of developing enhancement of allergen induced hypersensitivity reaction by airborne pollutants.

20. A method for the treatment and prevention of enhancement of allergen induced hypersensitivity reaction by airborne pollutants in a subject where the subject is administered a nucleic acid sequence selected from the group consisting of GSTM1 coding sequence or GSTP1 105val coding sequence.

21. A method for detecting a biological effect in subjects exposed to air pollutants comprising genotyping said subjects for polymorphisms within a group of gene comprising GSTM1 and GSTP1.

22. A method for detecting a modified biological effect in response to exposure to air pollutants in subjects with a GSTM1 null genotype compared to the biological effect detected in subject with a GSTM1 present genotype


SEQUENCE LISTING (16 sequences)

SEQ ID NO: 1 - DNA, 22 bp, Artificial Sequence
  Primer for the GSTM1 Gene
cttggaggaactccctgaaaag

SEQ ID NO: 2 - DNA, 21 bp, Artificial Sequence
  Primer for the GSTM1 Gene
tggaacctccataacacgtga

SEQ ID NO: 3 - DNA, 20 bp, Artificial Sequence
  Probe for the GSTM1 Gene
aagcggccatggtttgcagg

SEQ ID NO: 4 - DNA, 21 bp, Artificial Sequence
  Primer for the GSTT1 Gene
gtgcaaacacctcctggagat

SEQ ID NO: 5 - DNA, 21 bp, Artificial Sequence
  Primer for the GSTT1 Gene
agtccttggccttcagaatga

SEQ ID NO: 6 - DNA, 19 bp, Artificial Sequence
  Probe for the GSTT1 Gene
atgctgcccatccctgccc

SEQ ID NO: 7 - DNA, 20 bp, Artificial Sequence
  Primer for the GSTP1 Gene
cctggtggacatggtgaatg

SEQ ID NO: 8 - DNA, 27 bp, Artificial Sequence
  Primers for the GSTP1 Gene
tgctcacaccatagttggtgtagatga

SEQ ID NO: 9 - DNA, 17 bp, Artificial Sequence
  Probe for the GSTP1 Gene
tgcaaatacatctccct

SEQ ID NO: 10 - DNA, 16 bp, Artificial Sequence
  Probe for the GSTP1 Gene
ctgcaaatacgtctcc

SEQ ID NO: 11 - DNA, 546 bp, Homo Sapiens
atgcccatgatactggggtactgggacatccgcgggctggcccacgccatccgcctgctcctggaatacacagactcaagctatgaggaaaagaagtacacgatgggggacgctcctgattatgacagaagccagtggctgaatgaaaaattcaagctgggcctggactttcccaatctgccctacttgattgatggggctcacaagatcacccagagcaacgccatcttgtgctacattgcccgcaagcacaacctgtgtggggagacagaagaggagaagattcgtgtggacattttggagaaccagaccatggacaaccatatgcagctgggcatgatctgctacaatccagaatttgagaaactgaagccaaagtacttggaggaactccctgaaaagctaaagctctactcagagtttctggggaagcggccatggtttgcaggaaacaagggcttggagaagatctctgcctacatgaagtccagccgcttcctcccaagacctgtgttctcaaagatggctgtctggggcaacaagtag

SEQ ID NO: 12 - protein, 181 aa, Homo Sapiens
MPMILGYWDIRGLAHAIRLLLEYTDSSYEEKKYTMGDAPDYDRSQWLNEKFKLGLDFPNLPYLIDGAHKITQSNAILCYIARKHNLCGETEEEKIRVDILENQTMDNHMQLGMICYNPEFEKLKPKYLEELPEKLKLYSEFLGKRPWFAGNKGLEKISAYMKSSRFLPRPVFSKMAVWGNK

SEQ ID NO: 13 - DNA, 657 bp, Homo Sapiens
atgcccatgatactggggtactgggacatccgcgggctggcccacgccatccgcctgctcctggaatacacagactcaagctatgaggaaaagaagtacacgatgggggacgctcctgattatgacagaagccagtggctgaatgaaaaattcaagctgggcctggactttcccaatctgccctacttgattgatggggctcacaagatcacccagagcaacgccatcttgtgctacattgcccgcaagcacaacctgtgtggggagacagaagaggagaagattcgtgtggacattttggagaaccagaccatggacaaccatatgcagctgggcatgatctgctacaatccagaatttgagaaactgaagccaaagtacttggaggaactccctgaaaagctaaagctctactcagagtttctggggaagcggccatggtttgcaggaaacaagatcacttttgtagattttctcgtctatgatgtccttgacctccaccgtatatttgagcccaagtgcttggacgccttcccaaatctgaaggacttcatctcccgctttgagggcttggagaagatctctgcctacatgaagtccagccgcttcctcccaagacctgtgttctcaaagatggctgtctggggcaacaagtag

SEQ ID NO: 14 - protein, 218 aa, Homo Sapiens
MPMILGYWDIRGLAHAIRLLLEYTDSSYEEKKYTMGDAPDYDRSQWLNEKFKLGLDFPNLPYLIDGAHKITQSNAILCYIARKHNLCGETEEEKIRVDILENQTMDNHMQLGMICYNPEFEKLKPKYLEELPEKLKLYSEFLGKRPWFAGNKITFVDFLVYDVLDLHRIFEPKCLDAFPNLKDFISRFEGLEKISAYMKSSRFLPRPVFSKMAVWGNK

SEQ ID NO: 15 - DNA, 737 bp, Homo Sapiens
ggagtttcgccgccgcagtcttcgccaccatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagcgcccggcccaagctcaaggccttcctggcctcccctgagtacgtgaacctccccatcaatggcaacgggaaacagtgagggttggggggactctgagcgggaggcagagtttgccttcctttctccaggaccaataaaatttctaagagagct

SEQ ID NO: 16 - protein, 210 aa, Homo Sapiens
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ